(19) United States
(12) Patent Application Publication
(10) Pub. No.: US 2021/0113561 A1
(43) Pub. Date: Apr. 22, 2021

(54) PHARMACEUTICAL COMPOSITION, METHODS FOR TREATING AND USES THEREOF

(71) Applicant: Boehringer Ingelheim International GmbH, Ingelheim am Rhein (DE)

(72) Inventors: Joerg RIPPMANN, Schemmerhofen (DE); Thomas KLEIN, Radolfzell (DE); Michael MARK, Biberach an der Riss (DE); Eric Willams MAYOUX, Neauphle-le-Vieux (FR)

(21) Appl. No.: 17/047,708

(22) PCT Filed: Apr. 11, 2019
(86) PCT No.: PCT/EP2019/059341

§ 371 Date: Oct. 15, 2020

(30) Foreign Application Priority Data
Apr. 17, 2018 (EP) 18167739.4

(51) Int. Cl. A61K 31/506; A61K 45/06; A61P 3/10

(52) U.S. Cl. CPC A61K 31/506; A61P 3/10; A61K 45/06

(57) ABSTRACT

The invention relates to a pharmaceutical combination according to the invention comprising an AOC3 inhibitor according to the formula (I) wherein R¹ and A are as defined herein, and an SGLT2 inhibitor. In addition the present invention relates to methods for preventing, slowing the progression of, delaying or treating fibrotic disorders, metabolic disorders, inflammation disorders, ocular diseases, neuroinflammatory disorders or cancer in a patient in need thereof characterized in that the pharmaceutical combination according to the invention is administered to the patient.

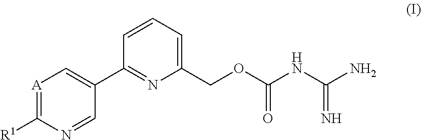

### FIELD OF THE INVENTION

The invention relates to a pharmaceutical combination and a pharmaceutical composition comprising an AOC3 inhibitor of formula (I) as defined hereinafter and an SGLT2-inhibitor.

Furthermore the invention relates to methods for treating or preventing a fibrotic disease, metabolic disease, an inflammatory disease, an ocular disease, a neuroinflammatory disease or cancer in a patient in need thereof characterized in that the pharmaceutical combination or composition is administered to the patient. In addition the invention relates to uses of the pharmaceutical combination or composition in a method for treating or preventing a disease as described hereinbefore or hereinafter.

### BACKGROUND OF THE INVENTION

The enzymatic activity of AOC3 (amine oxidase, copper containing 3; vascular adhesion protein 1) has been described already in 1967 as a monoamine oxidase activity in the plasma of chronic liver disease patients (Gressner, A. M. et al., 1982, J. Clin. Chem. Clin. Biochem. 20: 509-514; McEwen, C. M., Jr. et al., 1967, J. Lab Clin. Med. 70: 36-47). AOC3 has two closely homologous genes in the human genome: AOC1 which corresponds to a diamine oxidase (Chassande, O. et al., 1994, J. Biol. Chem. 269: 14484-14489) and AOC2, a SSAO with a specific expression in the retina (Imamura, Y. et al., 1997, Genomics 40: 277-283). AOC4 is a sequence that does not lead to a functional gene product in humans due to an internal stop-codon (Schwelberger, H. G., 2007, J. Neural Transm. 114: 757-762).

The enzyme contains an oxidized 2,4,5-trihydroxy-phenylalaninequinone (TPQ) and a copper ion in the active side. This characteristic catalytic center classifies the semi-carbazide-sensitive amine oxidase (SSAO, copper-containing amine:oxygen oxido-reductase (deaminating)): The type II membrane protein belongs to the family of copper containing amine oxidases together with several other diamine and the lysyl oxidases. However the later enzymes can be distinguished from AOC3 in their preference for diamines and the low sensitivity towards semicarbazide inhibition (Dunkel, P. et al., 2008, Curr. Med. Chem. 15: 1827-1839). On the other hand, monoamine oxidases contain the flavin adenine dinucleotide (FAD) cofactor in their reactive center like monoamine oxidase A (MAO-A) and monoamine oxidase B (MAO-B) and follow therefore a different reaction scheme.

AOC3 inhibitors represent a novel class of agents that are being developed for the treatment or improvement in a variety of indications, including inflammatory and fibrotic diseases, such as, for example NASH (non-alcoholic steatohepatitis), pulmonary fibrosis, retinopathy and nephropathy. For example, AOC3 inhibitors and their uses are disclosed in WO 2017/194453.

SGLT2 inhibitors represent a class of agents for the treatment of diabetes, in particular for the improvement of glycemic control in patients with type 2 diabetes mellitus. For example, SGLT2 inhibitors and their uses are disclosed in WO 2001/27128 and WO 2005/092877.

### SUMMARY OF THE INVENTION

The present invention relates to the use of an AOC3 inhibitor of formula (I) as defined hereinbefore or hereinafter for the manufacture of a medicament for use in a method as described hereinbefore and hereinafter.

In addition, the present invention relates to the use of an SGLT2 inhibitor for the manufacture of a medicament for use in a method as described hereinbefore and hereinafter.

The invention also relates to a use of a pharmaceutical combination or composition according to this invention for the manufacture of a medicament for use in a method as described hereinbefore and hereinafter.

One aim of the present invention is to provide a pharmaceutical combination or pharmaceutical composition and a method for preventing, slowing progression of, delaying, or treating of a fibrotic disease.

Another aim of the present invention is to provide a pharmaceutical combination or pharmaceutical composition and a method for preventing, slowing the progression of, delaying or treating of a metabolic disease.

A further aim of the present invention is to provide a pharmaceutical combination or pharmaceutical composition and a method for preventing, slowing progression of, delaying or treating of an inflammatory disease.

Another aim of the present invention is to provide a pharmaceutical combination or pharmaceutical composition and a method for preventing, slowing the progression of, delaying or treating of an ocular disease.

Another aim of the present invention is to provide a pharmaceutical combination or pharmaceutical composition and a method for preventing, slowing progression of, delaying or treating of a neuroinflammatory disease.

A further aim of the present invention is to provide a pharmaceutical combination or pharmaceutical composition and a method for preventing, slowing progression of, delaying or treating of a cancer.

Further aims of the present invention become apparent to the one skilled in the art by description hereinbefore and in the following and by the examples.

In one embodiment, the invention relates to a pharmaceutical combination or pharmaceutical composition comprising an AOC3 inhibitor of formula (I) as defined hereinafter and an SGLT2-inhibitor as defined hereinafter for preventing, slowing progression of, delaying or treating of one or more fibrotic diseases.

In another embodiment, the invention relates to a pharmaceutical combination or pharmaceutical composition comprising an AOC3 inhibitor of formula (I) as defined hereinafter and an SGLT2-inhibitor as defined hereinafter for preventing, slowing progression of, delaying or treating of a metabolic disease.

Moreover, another embodiment of the invention relates to a pharmaceutical combination or pharmaceutical composition comprising an AOC3 inhibitor of formula (I) as defined hereinafter and an SGLT2-inhibitor as defined hereinafter for preventing, slowing progression of, delaying or treating of an inflammatory disease.

Moreover, another embodiment of the invention relates to a pharmaceutical combination or pharmaceutical composition comprising an AOC3 inhibitor of formula (I) as defined hereinafter and an SGLT2-inhibitor as defined hereinafter for preventing, slowing progression of, delaying or treating of an ocular disease.

Moreover, another embodiment of the invention relates to a pharmaceutical combination or pharmaceutical composition comprising an AOC3 inhibitor of formula (I) as defined hereinafter and an SGLT2-inhibitor as defined hereinafter for preventing, slowing progression of, delaying or treating of neuroinflammatory disorders

Moreover, another embodiment of the invention relates to a pharmaceutical combination or pharmaceutical composition comprising an AOC3 inhibitor of formula (I) as defined hereinafter and an SGLT2-inhibitor as defined hereinafter for preventing, slowing progression of, delaying or treating of a cancer.

Therefore, in a first aspect the present invention provides a pharmaceutical combination or pharmaceutical composition comprising

(a) an AOC3 inhibitor of formula (I):

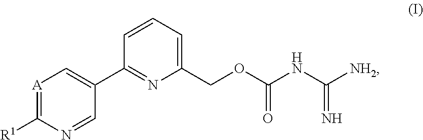

wherein:

A is selected from the group A-G1 consisting of: N and CH;

R¹ is selected from the group R¹-G1 consisting of:
C₁₋₆-alkyl, C₃₋₆-cycloalkyl, heterocyclyl, —O—R², —S—R², —NH—R² and —N(R²)₂,
   wherein each R² is independently selected from the group R²-G1 consisting of C₁₋₆-alkyl, C₃₋₆-cycloalkyl, heterocyclyl, —(C₁₋₂-alkyl)-(C₃₋₆-cycloalkyl), —(C₁₋₂-alkyl)-heterocyclyl, —(C₁₋₂-alkyl)-aryl, —(C₁₋₂-alkyl)-heteroaryl and —(C₁₋₂-alkyl)-C≡CH;
      wherein each heterocyclyl of R¹ and R² is a 4- to 7-membered saturated carbocyclic group, in which 1 or 2 CH₂-moieties are independently of each other replaced by an atom or group selected from NH, O, S, —S(═O)—, —S(═O)₂— or —C(═O)—; and
      wherein each aryl is selected from the group consisting of phenyl and naphthyl; and
      wherein each heteroaryl is a 5- or 6-membered heteroaromatic ring which contains 1, 2 or 3 heteroatoms independently selected from ═N—, —NH—, —O— and —S—, wherein in heteroaromatic groups containing a —CH═N— unit, this group is optionally replaced by —NH—C(═O)—; and
      wherein each alkyl, cycloalkyl, heterocyclyl, aryl or heteroaryl group of R¹ and R² is optionally independently substituted with one or more F, Cl, CN, OH, C₁₋₃-alkyl, —O—(C₁₋₃-alkyl), —C(═O)—(C₁₋₃-alkyl) and —C(═O)—(C₃₋₇-cycloalkyl);

wherein each of the above-mentioned alkyl and —O-alkyl groups may be linear or branched and are optionally substituted by one or more F;

a tautomer or stereoisomer thereof,

or a salt thereof, in particular a pharmaceutically acceptable salt thereof,

or a solvate or hydrate thereof,

and

(b) an SGLT2 inhibitor.

According to another aspect of the invention, there is provided a method of treating a disease associated with or modulated by AOC3, characterized in that an AOC3 inhibitor of formula (I) as defined hereinbefore and hereinafter and an SGLT2-inhibitor are administered, for example in combination or alternation, to the patient.

According to another aspect of the invention, there is provided a pharmaceutical combination or pharmaceutical composition for use in a method for preventing, slowing progression of, delaying or treating of one or more fibrotic, metabolic, inflammatory, ocular, neuroinflammatory diseases or cancers in a patient in need thereof.

According to another aspect of the invention there is provided the use of an AOC3 inhibitor of formula (I) as defined hereinbefore and hereinafter for the manufacture of a medicament for preventing, slowing progression of, delaying or treating of one or more fibrotic, metabolic, inflammatory, ocular, neuroinflammatory diseases or cancers in a patient in need thereof characterized in that the AOC3 inhibitor of formula (as defined hereinbefore and hereinafter is administered, for example in combination or alternation, with an SGLT2-inhibitor to the patient.

According to another aspect of the invention there is provided the use of an SGLT2 inhibitor for the manufacture of a medicament for preventing, slowing progression of, delaying or treating of one or more fibrotic, metabolic, inflammatory, ocular, neuroinflammatory diseases or cancers in a patient in need thereof characterized in that the SGLT2 inhibitor is administered, for example in combination or alternation, with an AOC3 inhibitor of formula (I) as defined hereinbefore and hereinafter to the patient.

According to another aspect of the invention, there is provided the use of a pharmaceutical combination or pharmaceutical composition according to the present invention for the manufacture of a medicament for preventing, slowing progression of, delaying or treating of one or more fibrotic, metabolic, inflammatory, ocular, neuroinflammatory diseases or cancers.

### DETAILED DESCRIPTION OF THE INVENTION

### Definitions

The following definitions may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The term “substituted” as used herein, means that any one or more hydrogens on the designated atom, radical or moiety is replaced with a selection from the indicated group, provided that the atom's normal valence is not exceeded, and that the substitution results in an acceptably stable compound.

In the groups, radicals, or moieties defined below, the number of carbon atoms is often specified preceding the group, for example, C₁₋₆-alkyl means an alkyl group or radical having 1 to 6 carbon atoms. In general, for groups comprising two or more subgroups, the last named subgroup is the radical attachment point, for example, the substituent “aryl-C₁₋₃-alkyl-” means an aryl group which is bound to a C₁₋₃-alkyl-group, the latter of which is bound to the core or to the group to which the substituent is attached.

In case a compound of the present invention is depicted in form of a chemical name and as a formula in case of any discrepancy the formula shall prevail.

An asterisk is may be used in sub-formulas to indicate the bond which is connected to the core molecule as defined.

The numeration of the atoms of a substituent starts with the atom which is closest to the core or to the group to which the substituent is attached.

For example, the term “3-carboxypropyl-group” represents the following substituent:

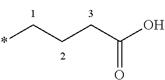 [SMILES: *CCCC(=O)O]

wherein the carboxy group is attached to the third carbon atom of the propyl group. The terms “1-methylpropyl-”, “2,2-dimethylpropyl-” or “cyclopropylmethyl-” group represent the following groups:

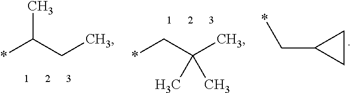

The asterisk may be used in sub-formulas to indicate the bond which is connected to the core molecule as defined.

In a definition of a group the term “wherein each X, Y and Z group is optionally substituted with” and the like denotes that each group X, each group Y and each group Z either each as a separate group or each as part of a composed group may be substituted as defined. For example a definition “Rex denotes H, C₁₋₃-alkyl, C₃₋₆-cycloalkyl, C₃₋₆-cycloalkyl-C₁₋₃-alkyl or C₁₋₃-alkyl-O—, wherein each alkyl group is optionally substituted with one or more L^{ex}.” or the like means that in each of the beforementioned groups which comprise the term alkyl, i.e. in each of the groups C₁₋₃-alkyl, C₃₋₆-cycloalkyl-C₁₋₃-alkyl and C₁₋₃-alkyl-O—, the alkyl moiety may be substituted with L^{ex} as defined.

In the following the term bicyclic includes spirocyclic.

Unless specifically indicated, throughout the specification and the appended claims, a given chemical formula or name shall encompass tautomers and all stereo, optical and geometrical isomers (e.g. enantiomers, diastereomers, E/Z isomers etc . . . ) and racemates thereof as well as mixtures in different proportions of the separate enantiomers, mixtures of diastereomers, or mixtures of any of the foregoing forms where such isomers and enantiomers exist, as well as salts, including pharmaceutically acceptable salts thereof and solvates thereof such as for instance hydrates including solvates of the free compounds or solvates of a salt of the compound.

The term halogen generally denotes fluorine, chlorine, bromine and iodine.

The term “C₁₋ₙ-alkyl”, wherein n is an integer from 1 to n, either alone or in combination with another radical denotes an acyclic, saturated, branched or linear hydrocarbon radical with 1 to n C atoms. For example the term C₁₋₅-alkyl embraces the radicals H₃C—, H₃C—CH₂—, H₃C—CH₂—CH₂—, H₃C—CH(CH₃)—, H₃C—CH₂—CH₂—CH₂—, H₃C—CH₂—CH(CH₃)—, H₃C—CH(CH₃)—CH₂—, H₃C—C(CH₃)₂—, H₃C—CH₂—CH₂—CH₂—CH₂—, H₃C—CH₂—CH₂—CH(CH₃)—, H₃C—CH₂—CH(CH₃)—CH₂—, H₃C—CH(CH₃)—CH₂—CH₂—, H₃C—CH₂—C(CH₃)₂—, H₃C—C(CH₃)₂—CH₂—, H₃C—CH(CH₃)—CH(CH₃)— and H₃C—CH₂—CH(CH₂CH₃)—.

The term “C₃₋ₙ-cycloalkyl”, wherein n is an integer 4 to n, either alone or in combination with another radical denotes a cyclic, saturated, unbranched hydrocarbon radical with 3 to n C atoms. The cyclic group may be mono-, bi-, tri- or spirocyclic, most preferably monocyclic. Examples of such cycloalkyl groups include cyclopropyl, cyclobutyl, cyclopentyl, cyclohexyl, cycloheptyl, cyclooctyl, cyclononyl, cyclododecyl, bicyclo[3.2.1.]octyl, spiro[4.5]decyl, norpinyl, norbonyl, norcaryl, adamantyl, etc.

Many of the terms given above may be used repeatedly in the definition of a formula or group and in each case have one of the meanings given above, independently of one another.

All rests and substituents as defined hereinbefore and hereinafter may be substituted with one or more F atoms.

All references cited in this application are specifically incorporated by cross-reference in their entirety. Reference to any such documents should not be construed as an admission that the document forms part of the common general knowledge or is prior art.

In the context of this specification the term “administering” and variations of that term including “administer” and “administration”, includes contacting, applying, delivering or providing a compound or composition of the invention to an organism, or a surface by any appropriate means. In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

In the context of this specification the term “effective amount” includes within its meaning a sufficient but non-toxic amount of a compound or composition of the invention to provide a desired effect. Thus, the term “therapeutically effective amount” includes within its meaning a sufficient but non-toxic amount of a compound or composition of the invention to provide the desired therapeutic effect. The exact amount required will vary from subject to subject depending on factors such as the species being treated, the sex, age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art.

The term “active ingredient” of a pharmaceutical composition according to the present invention means the AOC3 inhibitor and/or SGLT2 inhibitor according to the present invention.

The term “AOC3” in the scope of the present invention relates to amine oxidase, copper containing 3; also known as vascular adhesion protein 1 (VAP-1), also known as semicarbazide-sensitive amine oxidase (SSAO) enzyme, as primary amine oxidase, plasma amine oxidase and benzylamine oxidase. The enzyme contains an oxidized 2,4,5-trihydroxy-phenylalaninequinone (TPQ) and a copper ion in the active side. This characteristic catalytic center classifies the semi-carbazide-sensitive amine oxidase (SSAO, copper-containing amine:oxygen oxido-reductase (deaminating)): The type II membrane protein belongs to the family of copper containing amine oxidases together with several other diamine and the lysyl oxidases. However the later enzymes can be distinguished from AOC3 in their preference for diamines and the low sensitivity towards semicarbazide inhibition (Dunkel, P. et al., 2008, Curr. Med. Chem. 15: 1827-1839). In the scope of the present invention, AOC3 is used to describe the semicarbazide-sensitive amine oxidase (SSAO) enzyme.

The term “AOC3 inhibitor” in the scope of the present invention relates to a compound, in particular to a pyridinyl derivative of formula (I), that exhibits an inhibitory effect on the AOC3 enzyme, in particular on the human AOC3. The inhibitory effect on AOC3 measured as IC₅₀ is for example below 5000 nM, preferably below 1000 nM, more preferably below 300 nM and most preferably below 100 nM. The inhibitory effect on AOC3 can be determined by methods known in the literature, in particular as described in the application WO 2017/194453 (pages 25/28), which are incorporated herein by reference in its entirety. The term “AOC3 inhibitor” also comprises any pharmaceutically acceptable salts thereof, hydrates and solvates thereof, including the respective crystalline forms or polymorphs.

The term “SGLT2 inhibitor” in the scope of the present invention relates to a compound which shows an inhibitory effect on the sodium-glucose transporter 2 (SGLT2), in particular the human SGLT2. The inhibitory effect on hSGLT2 measured as IC50 is prerably below 1000 nM, even more preferably below 100 nM, most preferably below 50 nM. IC50 values of SGLT2 inhibitors are usually above 0.01 nM, or even equal to or above 0.1 nM. The inhibitory effect on hSGLT2 can be determined by methods known in the literature, in particular as described in the application WO 2005/092877 or WO 2007/093610 (pages 23/24), which are incorporated herein by reference in its entirety. The term “SGLT2 inhibitor” also comprises any pharmaceutically acceptable salts thereof, hydrates and solvates thereof, including the respective crystalline forms.

The terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present invention may be used for instance as therapeutic treatment over a period of time as well as for chronic therapy.

The terms “prophylactically treating”, “preventivally treating” and “preventing” are used interchangeably and comprise a treatment of patients at risk to develop a condition mentioned hereinbefore, thus reducing said risk.

The term “body mass index” or “BMI” of a human patient is defined as the weight in kilograms divided by the square of the height in meters, such that BMI has units of kg/m².

The term “overweight” is defined as the condition wherein the individual has a BMI greater than or 25 kg/m² and less than 30 kg/m². The terms “overweight” and “pre-obese” are used interchangeably.

The terms “obesity” or “being obese” and the like are defined as the condition wherein the individual has a BMI equal to or greater than 30 kg/m². According to a WHO definition the term obesity may be categorized as follows: the term “class I obesity” is the condition wherein the BMI is equal to or greater than 30 kg/m² but lower than 35 kg/m²; the term “class II obesity” is the condition wherein the BMI is equal to or greater than 35 kg/m² but lower than 40 kg/m²; the term “class Ill obesity” is the condition wherein the BMI is equal to or greater than 40 kg/m².

The indication obesity includes in particular exogenic obesity, hyperinsulinaemic obesity, hyperplasmic obesity, hyperphyseal adiposity, hypoplasmic obesity, hypothyroid obesity, hypothalamic obesity, symptomatic obesity, infantile obesity, upper body obesity, alimentary obesity, hypogonadal obesity, central obesity, visceral obesity, abdominal obesity.

The term “visceral obesity” is defined as the condition wherein a waist-to-hip ratio of greater than or equal to 1.0 in men and 0.8 in women is measured. It defines the risk for insulin resistance and the development of pre-diabetes.

The term “abdominal obesity” is usually defined as the condition wherein the waist circumference is >40 inches or 102 cm in men, and is >35 inches or 94 cm in women. With regard to a Japanese ethnicity or Japanese patients abdominal obesity may be defined as waist circumference 85 cm in men and 90 cm in women (see e.g. investigating committee for the diagnosis of metabolic syndrome in Japan).

The term “euglycemia” is defined as the condition in which a subject has a fasting blood glucose concentration within the normal range, greater than 70 mg/dL (3.89 mmol/L) and less than 100 mg/dL (5.6 mmol/L). The word “fasting” has the usual meaning as a medical term.

The term “hyperglycemia” is defined as the condition in which a subject has a fasting blood glucose concentration above the normal range, greater than 100 mg/dL (5.6 mmol/L). The word “fasting” has the usual meaning as a medical term.

The term “hypoglycemia” is defined as the condition in which a subject has a blood glucose concentration below the normal range, in particular below 70 mg/dL (3.89 mmol/L).

The term “postprandial hyperglycemia” is defined as the condition in which a subject has a 2 hour postprandial blood glucose or serum glucose concentration greater than 200 mg/dL (11.11 mmol/L).

The term “impaired fasting blood glucose” or “IFG” is defined as the condition in which a subject has a fasting blood glucose concentration or fasting serum glucose concentration in a range from 100 to 125 mg/dl (i.e. from 5.6 to 6.9 mmol/I), in particular greater than 110 mg/dL and less than 126 mg/dl (7.00 mmol/L). A subject with “normal fasting glucose” has a fasting glucose concentration smaller than 100 mg/dl, i.e. smaller than 5.6 mmol/I.

The term “impaired glucose tolerance” or “IGT” is defined as the condition in which a subject has a 2 hour postprandial blood glucose or serum glucose concentration greater than 140 mg/dl (7.78 mmol/L) and less than 200 mg/dL (11.11 mmol/L). The abnormal glucose tolerance, i.e. the 2 hour postprandial blood glucose or serum glucose concentration can be measured as the blood sugar level in mg of glucose per dL of plasma 2 hours after taking 75 g of glucose after a fast. A subject with “normal glucose tolerance” has a 2 hour postprandial blood glucose or serum glucose concentration smaller than 140 mg/dl (7.78 mmol/L).

The term “hyperinsulinemia” is defined as the condition in which a subject with insulin resistance, with or without euglycemia, has fasting or postprandial serum or plasma insulin concentration elevated above that of normal, lean individuals without insulin resistance, having a waist-to-hip ratio <1.0 (for men) or <0.8 (for women).

The terms “insulin-sensitizing”, “insulin resistance-improving” or “insulin resistance-lowering” are synonymous and used interchangeably.

The term “insulin resistance” is defined as a state in which circulating insulin levels in excess of the normal response to a glucose load are required to maintain the euglycemic state (Ford E S, et al. JAMA. (2002) 287:356-9). A method of determining insulin resistance is the euglycaemic-hyperinsulinaemic clamp test. The ratio of insulin to glucose is determined within the scope of a combined insulin-glucose infusion technique. There is found to be insulin resistance if the glucose absorption is below the 25th percentile of the background population investigated (WHO definition). Rather less laborious than the clamp test are so called minimal models in which, during an intravenous glucose tolerance test, the insulin and glucose concentrations in the blood are measured at fixed time intervals and from these the insulin resistance is calculated. With this method, it is not possible to distinguish between hepatic and peripheral insulin resistance.

Furthermore, insulin resistance, the response of a patient with insulin resistance to therapy, insulin sensitivity and hyperinsulinemia may be quantified by assessing the “homeostasis model assessment to insulin resistance (HOMA-IR)” score, a reliable indicator of insulin resistance (Katsuki A, et al. Diabetes Care 2001; 24: 362-5). Further reference is made to methods for the determination of the HOMA-index for insulin sensitivity (Matthews et al., Diabetologia 1985, 28: 412-19), of the ratio of intact proinsulin to insulin (Forst et al., Diabetes 2003, 52(Suppl. 1): A459) and to an euglycemic clamp study. In addition, plasma adiponectin levels can be monitored as a potential surrogate of insulin sensitivity. The estimate of insulin resistance by the homeostasis assessment model (HOMA)-IR score is calculated with the formula (Galvin P, et al. Diabet Med 1992; 9:921-8):

HOMA-IR=[fasting serum insulin (μU/mL)]×[fasting plasma glucose(mmol/L)]/22.5

Insulin resistance can be confirmed in these individuals by calculating the HOMA-IR score. For the purpose of this invention, insulin resistance is defined as the clinical condition in which an individual has a HOMA-IR score>4.0 or a HOMA-IR score above the upper limit of normal as defined for the laboratory performing the glucose and insulin assays.

As a rule, other parameters are used in everyday clinical practice to assess insulin resistance. Preferably, the patient's triglyceride concentration is used, for example, as increased triglyceride levels correlate significantly with the presence of insulin resistance.

Individuals likely to have insulin resistance are those who have two or more of the following attributes: 1) overweight or obese, 2) high blood pressure, 3) hyperlipidemia, 4) one or more 1^{st} degree relative with a diagnosis of IGT or IFG or type 2 diabetes.

Patients with a predisposition for the development of IGT or IFG or type 2 diabetes are those having euglycemia with hyperinsulinemia and are by definition, insulin resistant. A typical patient with insulin resistance is usually overweight or obese. If insulin resistance can be detected, this is a particularly strong indication of the presence of pre-diabetes. Thus, it may be that in order to maintain glucose homoeostasis a person needs 2-3 times as much insulin as a healthy person, without this resulting in any clinical symptoms.

“Pre-diabetes” is a general term that refers to an intermediate stage between normal glucose tolerance (NGT) and overt type 2 diabetes mellitus (T2DM), also referred to as intermediate hyperglycaemia. Therefore in one aspect of the present invention “pre-diabetes” is diagnosed in an individual if HbA1c is more or equal to 5.7% and less than 6.5%. According to another aspect of this invention “pre-diabetes” represents 3 groups of individuals, those with impaired glucose tolerance (IGT) alone, those with impaired fasting glucose (IFG) alone or those with both IGT and IFG. IGT and IFG usually have distinct pathophysiologic etiologies, however also a mixed condition with features of both can exist in patients. Therefore in another aspect of the present invention a patient being diagnosed of having “pre-diabetes” is an individual with diagnosed IGT or diagnosed IFG or diagnosed with both IGT and IFG. Following the definition according to the American Diabetes Association (ADA) and in the context an aspect of the present invention a patient being diagnosed of having “pre-diabetes” is an individual with:

a) a fasting plasma glucose (FPG) concentration<100 mg/dL [1 mg/dL=0.05555 mmol/L] and a 2-hour plasma glucose (PG) concentration, measured by a 75-g oral glucose tolerance test (OGTT), ranging between ≥140 mg/dL and <200 mg/dL (i.e., IGT); or
b) a fasting plasma glucose (FPG) concentration between ≥100 mg/dL and <126 mg/dL and a 2-hour plasma glucose (PG) concentration, measured by a 75-g oral glucose tolerance test (OGTT) of <140 mg/dL (i.e., IFG); or
c) a fasting plasma glucose (FPG) concentration between ≥100 mg/dL and <126 mg/dL and a 2-hour plasma glucose (PG) concentration, measured by a 75-g oral glucose tolerance test (OGTT), ranging between ≥140 mg/dL and <200 mg/dL (i.e., both IGT and IFG).

Patients with “pre-diabetes” are individuals being pre-disposed to the development of type 2 diabetes. Pre-diabetes extends the definition of IGT to include individuals with a fasting blood glucose within the high normal range ≥100 mg/dL (J. B. Meigs, et al. Diabetes 2003; 52:1475-1484). The scientific and medical basis for identifying pre-diabetes as a serious health threat is laid out in a Position Statement entitled “The Prevention or Delay of Type 2 Diabetes” issued jointly by the American Diabetes Association and the National Institute of Diabetes and Digestive and Kidney Diseases (Diabetes Care 2002; 25:742-749).

The methods to investigate the function of pancreatic beta-cells are similar to the above methods with regard to insulin sensitivity, hyperinsulinemia or insulin resistance: An improvement of beta-cell function can be measured for example by determining a HOMA-index (homeostasis model assessment) for beta-cell function, HOMA-B, (Matthews et al., Diabetologia 1985, 28: 412-19), the ratio of intact proinsulin to insulin (Forst et al., Diabetes 2003, 52(Suppl. 1): A459), first and second phase insulin secretion after an oral glucose tolerance test or a meal tolerance test (Stumvoll et al., Diabetes care 2000, 23: 295-301), the insulin/C-peptide secretion after an oral glucose tolerance test or a meal tolerance test, or by employing a hyperglycemic clamp study and/or minimal modeling after a frequently sampled intravenous glucose tolerance test (Stumvoll et al., Eur J Clin Invest 2001, 31: 380-81).

The term “type 1 diabetes” is defined as the condition in which a subject has, in the presence of autoimmunity towards the pancreatic beta-cell or insulin, a fasting blood glucose or serum glucose concentration greater than 125 mg/dL (6.94 mmol/L). If a glucose tolerance test is carried out, the blood sugar level of a diabetic will be in excess of 200 mg of glucose per dL (11.1 mmol/l) of plasma 2 hours after 75 g of glucose have been taken on an empty stomach, in the presence of autoimmunity towards the pancreatic beta cell or insulin. In a glucose tolerance test 75 g of glucose are administered orally to the patient being tested after 10-12 hours of fasting and the blood sugar level is recorded immediately before taking the glucose and 1 and 2 hours after taking it. The presence of autoimmunity towards the pancreatic beta-cell may be observed by detection of circulating islet cell autoantibodies [“type 1A diabetes mellitus”], i.e., at least one of: GAD65 [glutamic acid decarboxylase-65], ICA [islet-cell cytoplasm], IA-2 [intracytoplasmatic domain of the tyrosine phosphatase-like protein IA-2], ZnT8 [zinc-transporter-8] or anti-insulin; or other signs of autoimmunity without the presence of typical circulating autoantibodies [type 1B diabetes], i.e. as detected through pancreatic biopsy or imaging). Typically a genetic predisposition is present (e.g. HLA, INS VNTR and PTPN22), but this is not always the case.

The term “type 2 diabetes mellitus” or “T2DM” is defined as the condition in which a subject has a fasting blood glucose or serum glucose concentration greater than 125 mg/dL (6.94 mmol/L). The measurement of blood glucose values is a standard procedure in routine medical analysis. If a glucose tolerance test is carried out, the blood sugar level of a diabetic will be in excess of 200 mg of glucose per dL (11.1 mmol/l) of plasma 2 hours after 75 g of glucose have been taken on an empty stomach. In a glucose tolerance test 75 g of glucose are administered orally to the patient being tested after 10-12 hours of fasting and the blood sugar level is recorded immediately before taking the glucose and 1 and 2 hours after taking it. In a healthy subject, the blood sugar level before taking the glucose will be between 60 and 110 mg per dL of plasma, less than 200 mg per dL 1 hour after taking the glucose and less than 140 mg per dL after 2 hours. If after 2 hours the value is between 140 and 200 mg, this is regarded as abnormal glucose tolerance.

The term “late stage type 2 diabetes mellitus” includes patients with a secondary drug failure, indication for insulin therapy and progression to micro- and macrovascular complications e.g. diabetic nephropathy, or coronary heart disease (CHD).

The term “HbA1c” refers to the product of a non-enzymatic glycation of the haemoglobin B chain. Its determination is well known to one skilled in the art. In monitoring the treatment of diabetes mellitus the HbA1c value is of exceptional importance. As its production depends essentially on the blood sugar level and the life of the erythrocytes, the HbA1c in the sense of a “blood sugar memory” reflects the average blood sugar levels of the preceding 4-6 weeks. Diabetic patients whose HbA1c value is consistently well adjusted by intensive diabetes treatment (i.e. <6.5% of the total haemoglobin in the sample), are significantly better protected against diabetic microangiopathy. For example, metformin on its own achieves an average improvement in the HbA1c value in the diabetic of the order of 1.0-1.5%. This reduction of the HbA1C value is not sufficient in all diabetics to achieve the desired target range of <7% or <6.5% and preferably <6% HbA1c.

The term “insufficient glycemic control” or “inadequate glycemic control” in the scope of the present invention means a condition wherein patients show HbA1c values above 6.5%, in particular above 7.0%, even more preferably above 7.5%, especially above 8%.

The “metabolic syndrome”, also called “syndrome X” (when used in the context of a metabolic disorder), also called the “dysmetabolic syndrome” is a syndrome complex with the cardinal feature being insulin resistance (Laaksonen D E, et al. Am J Epidemiol 2002; 156:1070-7). According to the ATP III/NCEP guidelines (Executive Summary of the Third Report of the National Cholesterol Education Program (NCEP) Expert Panel on Detection, Evaluation, and Treatment of High Blood Cholesterol in Adults (Adult Treatment Panel III) JAMA: Journal of the American Medical Association (2001) 285:2486-2497), diagnosis of the metabolic syndrome is made when three or more of the following risk factors are present:
1. Abdominal obesity, defined as waist circumference>40 inches or 102 cm in men, and >35 inches or 94 cm in women; or with regard to a Japanese ethnicity or Japanese patients defined as waist circumference≥85 cm in men and ≥90 cm in women;
2. Triglycerides: ≥150 mg/dL
3. HDL-cholesterol<40 mg/dL in men
4. Blood pressure≥130/85 mm Hg (SBP≥130 or DBP≥85)
5. Fasting blood glucose 100 mg/dL

The NCEP definitions have been validated (Laaksonen D E, et al. Am J Epidemiol. (2002) 156:1070-7). Triglycerides and HDL cholesterol in the blood can also be determined by standard methods in medical analysis and are described for example in Thomas L (Editor): “Labor und Diagnose”, TH-Books Verlagsgesellschaft mbH, Frankfurt/Main, 2000.

The aspects according to the present invention, in particular the pharmaceutical compositions, combinations, methods and uses, refer to an AOC3 inhibitor of formula (I) as defined hereinbefore and hereinafter, or a pharmaceutically acceptable salt thereof. Preferably the AOC3 inhibitor is selected from the group G1 consisting of compounds of the formula (I):

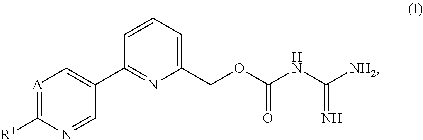

wherein:

A is selected from the group A-G1 consisting of: N and CH;

R¹ is selected from the group R¹-G1 consisting of:
C₁₋₆-alkyl, C₃₋₆-cycloalkyl, heterocyclyl, —O—R², —S—R², —NH—R² and —N(R²)₂,
   wherein each R² is independently selected from the group R²-G1 consisting of C₁₋₆-alkyl, C₃₋₆-cycloalkyl, heterocyclyl, —(C₁₋₂-alkyl)-(C₃₋₆-cycloalkyl), —(C₁₋₂-alkyl)-heterocyclyl, —(C₁₋₂-alkyl)-aryl, —(C₁₋₂-alkyl)-heteroaryl and —(C₁₋₂-alkyl)-C≡CH;
      wherein each heterocyclyl of R¹ and R² is a 4- to 7-membered saturated carbocyclic group, in which 1 or 2 CH₂-moieties are independently of each other replaced by an atom or group selected from NH, O, S, —S(═O)—, —S(═O)₂— or —C(═O)—; and
      wherein each aryl is selected from the group consisting of phenyl and naphthyl; and
      wherein each heteroaryl is a 5- or 6-membered heteroaromatic ring which contains 1, 2 or 3 heteroatoms independently selected from ═N—, —NH—, —O— and —S—, wherein in heteroaromatic groups containing a —CH═N— unit, this group is optionally replaced by —NH—C(═O)—; and
      wherein each alkyl, cycloalkyl, heterocyclyl, aryl or heteroaryl group of R¹ and R² is optionally independently substituted with one or more F, Cl, CN, OH, C₁₋₃-alkyl, —O—(C₁₋₃-alkyl), —C(═O)—(C₁₋₃-alkyl) and —C(═O)—(C₃₋₇-cycloalkyl);

wherein each of the above-mentioned alkyl and —O-alkyl groups may be linear or branched and are optionally substituted by one or more F;

a tautomer or stereoisomer thereof,

or a salt thereof, in particular a pharmaceutically acceptable salt thereof,

or a solvate or hydrate thereof.

Compounds of the formula (I) and methods of their synthesis are described in WO 2017/194453.

In the following embodiments of compounds according to the formula (I) are described:

Unless otherwise stated, the groups, residues, and substituents, particularly A, R¹ and R², are defined as above and hereinafter. If residues, substituents or groups occur several times in a compound, as for example R², they may have the same or different meanings. Some preferred meanings of individual groups and substituents of the compounds according to the invention will be given hereinafter. Any and each of these definitions may be combined with each other.

A:

A-G1:

The group A is preferably selected from the group A-G1 as defined above.

A-G2:

In another embodiment the group A is selected from the group A-G2 consisting of N.

A-G3:

In another embodiment the group A is selected from the group A-G3 consisting of CH.

R¹:

R¹-G1:

The group R¹ is preferably selected from the group R¹-G1 as defined above.

R¹-G2:

In one embodiment the group R¹ is selected from the group R¹-G2 consisting of:

C₁₋₄-alkyl, C₃₋₅-cycloalkyl, heterocyclyl, —O—R², —S—R², —NH—R² and —N(R²)₂;
wherein each heterocyclyl is a 4- to 6-membered saturated carbocyclic group, in which 1 or 2 CH₂-moieties are replaced by a heteroatom selected from NH, O or S; and
wherein each alkyl, cycloalkyl or heterocyclyl group is optionally independently substituted with 1 to 5 F and/or 1 to 3 substituents independently selected from the group consisting of Cl, CN, OH, C₁₋₂-alkyl, —O—(C₁₋₂-alkyl), —C(═O)—(C₁₋₂-alkyl) and —C(═O)—(C₃₋₄-cycloalkyl).

R¹-G3:

In another embodiment the group R¹ is selected from the group R¹-G3 consisting of:

C₁₋₃-alkyl, C₃₋₄-cycloalkyl, heterocyclyl, —O—R², —S—R², —NH—R² and —N(R²)₂;
wherein each heterocyclyl is selected from the group consisting of azetidinyl, piperidinyl, piperazinyl, tetrahydrofuranyl, tetrahydropyranyl and morpholinyl; and
wherein each alkyl, cycloalkyl or heterocyclyl group is optionally independently substituted with 1 to 3 F and/or one substituent selected from the group consisting of CN, OH, CH₃, —O—CH₃, —C(═O)—CH₃ and —C(═O)-cyclopropyl.

R¹-G4:

In another embodiment the group R¹ is selected from the group R¹-G4 consisting of:

C₁₋₂-alkyl, C₃₋₄-cycloalkyl, heterocyclyl, —O—R², —NH—R² and —N(R²)₂;
wherein each heterocyclyl is selected from the group consisting of azetidinyl, piperidinyl, tetrahydrofuranyl, tetrahydropyranyl and morpholinyl; and
wherein each alkyl, cycloalkyl or heterocyclyl group is optionally independently substituted with 1 to 3 F or one substituent selected from the group consisting of CN, OH, CH₃, —O—CH₃, —C(═O)—CH₃ and —C(═O)-cyclopropyl.

R¹-G5:

In another embodiment the group R¹ is selected from the group R¹-G5 consisting of: cyclopropyl, heterocyclyl and —O—R²;
wherein each heterocyclyl is selected from the group consisting of azetidinyl, piperidinyl, tetrahydrofuranyl, tetrahydropyranyl and morpholinyl; and
wherein each heterocyclyl group is optionally independently substituted with one substituent selected from the group consisting of F, CN, OH, CH₃, —O—CH₃.

R¹-G6:

In another embodiment the group R¹ is selected from the group R¹-G6 consisting of:
a) CH₃;
b) —O—C₁₋₄-alkyl optionally substituted with 1-3 F or one —OCH₃;
c) —O—C₂₋₄-alkyl terminally substituted with —C≡CH;
d) —S—CH₃;
e) cyclopropyl;
f) —NH—(C₁₋₃-alkyl) and —N(CH₃)(C₁₋₃-alkyl), wherein each alkyl group is optionally substituted with 1-3 F or one —OCH₃;
g) azetidinyl, tetrahydropyranyl and morpholinyl, each optionally substituted with —OCH₃;
h) tetrahydrofuranyloxy;
i) —O—CH₂—R³,
   wherein R³ is C₃₋₄-cycloalkyl optionally substituted with 1 or 2 substituents independently selected from the group consisting of F and CN; tetrahydropyranyl;
   piperidinyl optionally substituted with —C(═O)—CH₃ or —C(═O)-cyclopropyl; isoxazolyl, thiazolyl or thiadiazolyl;
j) —O—CH(CH₃)-oxazolyl;
k) —N(R^{N})—R⁴,
   wherein R^{N} is H or CH₃, and
   R⁴ is tetrahydrofuranyl, tetrahydropyranyl or —(CH₂)-isoxazolyl.

R¹-G7:

In another embodiment the group R¹ is selected from the group R¹-G7 consisting of:

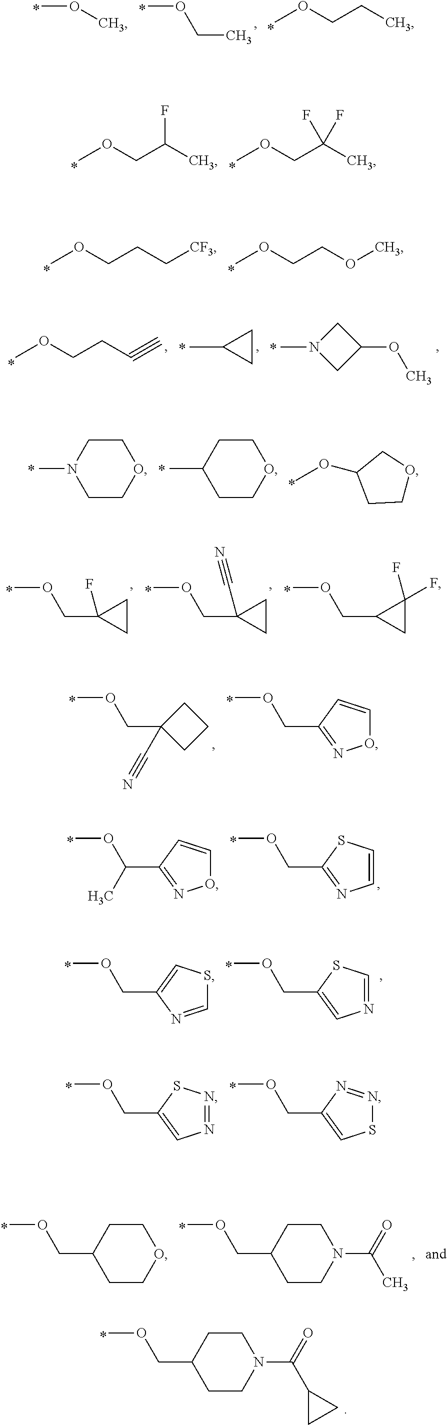

R²:

R²-G1:

The group R² is preferably selected from the group R²-G1 as defined above.

R²-G2:

In one embodiment the group R² is selected from the group R²-G2 consisting of

C₁₋₄-alkyl, C₃₋₅-cycloalkyl, heterocyclyl, —(C₁₋₂-alkyl)-(C₃₋₅-cycloalkyl), —(C₁₋₂-alkyl)-heterocyclyl, —(C₁₋₂-alkyl)-aryl, —(C₁₋₂-alkyl)-heteroaryl and —(C₁₋₂-alkyl)-C≡CH;
wherein each heterocyclyl is a 4- to 6-membered saturated carbocyclic group, in which 1 or 2 CH₂-moieties are replaced by a heteroatom selected from NH, O or S; and
wherein each aryl is selected from the group consisting of phenyl and naphthyl; and
wherein each heteroaryl is a 5- or 6-membered heteroaromatic ring which contains 1, 2 or 3 heteroatoms independently selected from ═N—, —NH—, —O— and —S—; and
wherein each alkyl, cycloalkyl, heterocyclyl, aryl or heteroaryl group is optionally independently substituted with one or more F, C, CN, OH, C₁₋₂-alkyl, —O—(C₁₋₂-alkyl), —C(═O)—(C₁₋₂-alkyl) and —C(═O)—(C₃₋₇-cycloalkyl).

R²-G3:

In another embodiment the group R² is selected from the group R²-G3 consisting of C₁₋₄-alkyl, C₃₋₄-cycloalkyl, heterocyclyl, —(C₁₋₂-alkyl)-(C₃₋₄-cycloalkyl), —(C₁₋₂-alkyl)-heterocyclyl, —(C₁₋₂-alkyl)-phenyl, —(C₁₋₂-alkyl)-heteroaryl and —(C₁₋₂-alkyl)-C≡CH;
wherein each heterocyclyl is selected from the group consisting of azetidinyl, tetrahydrofuranyl, tetrahydrofuranyl and piperidinyl; and
wherein each heteroaryl is selected from the group consisting of isoxazolyl, thiazolyl and thiadiazolyl; and
wherein each alkyl, cycloalkyl, heterocyclyl, aryl or heteroaryl group is optionally independently substituted with one or more F, CN, OH, CH₃, —OCH₃, —C(═O)—CH₃ and —C(═O)-cyclopropyl.

R²-G4:

In another embodiment the group R² is selected from the group R²-G4 consisting of C₁₋₄-alkyl, —CH₂—(C₃₋₄-cycloalkyl), —CH₂-heterocyclyl, —CH₂-heteroaryl and —CH₂—CH₂—C≡CH;
wherein each heterocyclyl is selected from the group consisting of tetrahydrofuranyl and piperidinyl; and
wherein each heteroaryl is selected from the group consisting of isoxazolyl, thiazolyl and thiadiazolyl; and
wherein each alkyl, cycloalkyl, heterocyclyl, aryl or heteroaryl group is optionally independently substituted with one or more F, CN, CH₃, —OCH₃, —C(═O)—CH₃ and —C(═O)-cyclopropyl.

R²-G5:

In another embodiment the group R² is selected from the group R²-G5 consisting of C₁₋₄-alkyl, —CH₂—(C₃₋₄-cycloalkyl), —CH₂-heteroaryl and —CH₂—CH₂—C≡CH;
wherein each heteroaryl is selected from the group consisting of isoxazolyl, thiazolyl and thiadiazolyl; and
wherein each alkyl, cycloalkyl, aryl or heteroaryl group is optionally independently substituted with one or more F, CN and —OCH₃.

Examples of preferred subgeneric embodiments of compounds of formula (I) are set forth in the following table 1, wherein each substituent group of each embodiment is defined according to the definitions set forth above:

TABLE 1

| No. | A | R¹ | R² |
|---|---|---|---|
| 1 | A-G1 | R¹-G1 | R²-G1 |
| 2 | A-G2 | R¹-G1 | R²-G1 |
| 3 | A-G1 | R¹-G1 | R²-G2 |
| 4 | A-G2 | R¹-G1 | R²-G2 |
| 5 | A-G1 | R¹-G1 | R²-G3 |
| 6 | A-G2 | R¹-G1 | R²-G3 |
| 7 | A-G1 | R¹-G1 | R²-G4 |
| 8 | A-G2 | R¹-G1 | R²-G4 |
| 9 | A-G1 | R¹-G1 | R²-G5 |
| 10 | A-G2 | R¹-G1 | R²-G5 |
| 11 | A-G1 | R¹-G2 | R²-G1 |
| 12 | A-G2 | R¹-G2 | R²-G1 |
| 13 | A-G1 | R¹-G2 | R²-G2 |
| 14 | A-G2 | R¹-G2 | R¹-G2 |
| 15 | A-G1 | R¹-G2 | R²-G3 |
| 16 | A-G2 | R¹-G2 | R²-G3 |
| 17 | A-G1 | R¹-G2 | R²-G4 |
| 18 | A-G2 | R¹-G2 | R²-G4 |
| 19 | A-G1 | R¹-G2 | R²-G5 |
| 20 | A-G2 | R¹-G2 | R²-G5 |
| 21 | A-G1 | R¹-G3 | R²-G1 |
| 22 | A-G2 | R¹-G3 | R²-G1 |
| 23 | A-G1 | R¹-G3 | R²-G2 |
| 24 | A-G2 | R¹-G3 | R²-G2 |
| 25 | A-G1 | R¹-G3 | R²-G3 |
| 26 | A-G2 | R¹-G3 | R²-G3 |
| 27 | A-G1 | R¹-G3 | R²-G4 |
| 28 | A-G2 | R¹-G3 | R²-G4 |
| 29 | A-G1 | R¹-G3 | R²-G5 |
| 30 | A-G2 | R¹-G3 | R²-G5 |
| 31 | A-G1 | R¹-G4 | R²-G1 |
| 32 | A-G2 | R¹-G4 | R²-G1 |
| 33 | A-G1 | R¹-G4 | R²-G2 |
| 34 | A-G2 | R¹-G4 | R²-G2 |
| 35 | A-G1 | R¹-G4 | R²-G3 |
| 36 | A-G2 | R¹-G4 | R²-G3 |
| 37 | A-G1 | R¹-G4 | R²-G4 |
| 38 | A-G2 | R¹-G4 | R²-G4 |
| 39 | A-G1 | R¹-G4 | R²-G5 |
| 40 | A-G2 | R¹-G4 | R²-G5 |
| 41 | A-G1 | R¹-G5 | R²-G1 |
| 42 | A-G2 | R¹-G5 | R²-G1 |
| 43 | A-G1 | R¹-G5 | R²-G2 |
| 44 | A-G2 | R¹-G5 | R²-G2 |
| 45 | A-G1 | R¹-G5 | R²-G3 |
| 46 | A-G2 | R¹-G5 | R²-G3 |
| 47 | A-G1 | R¹-G5 | R²-G4 |
| 48 | A-G2 | R¹-G5 | R²-G4 |
| 49 | A-G1 | R¹-G5 | R²-G5 |
| 50 | A-G2 | R¹-G5 | R²-G5 |
| 51 | A-G1 | R¹-G6 | — |
| 52 | A-G2 | R¹-G6 | — |
| 53 | A-G1 | R¹-G7 | — |
| 54 | A-G2 | R¹-G7 | — |
| 55 | A-G3 | R¹-G1 | R²-G1 |
| 56 | A-G3 | R¹-G2 | R²-G2 |
| 57 | A-G3 | R¹-G3 | R²-G2 |
| 58 | A-G3 | R¹-G3 | R²-G3 |
| 59 | A-G3 | R¹-G4 | R²-G3 |
| 60 | A-G3 | R¹-G4 | R²-G4 |
| 61 | A-G3 | R¹-G4 | R²-G5 |
| 62 | A-G3 | R¹-G5 | R²-G4 |
| 63 | A-G3 | R¹-G5 | R²-G5 |
| 64 | A-G3 | R¹-G6 | — |
| 65 | A-G3 | R¹-G7 | — |

The following preferred embodiments of compounds of the formula (I) are described using generic formulae (I.1) to (I.2), wherein any tautomers and stereoisomers, solvates, hydrates and salts thereof, in particular the pharmaceutically acceptable salts thereof, are encompassed.

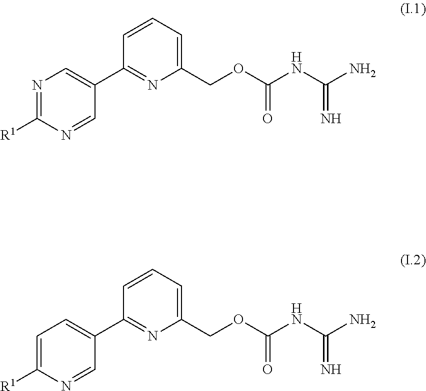

wherein in of the above formulae (I.1) to (I.2), the group R¹ is as defined above.

A preferred subgroup of compounds of formula (I) concerns compounds of formula

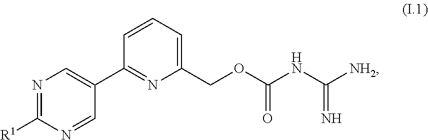

wherein

R¹ is selected from the group consisting of cyclopropyl, heterocyclyl and —O—R²;
wherein R² is selected from the group consisting of C₁₋₆-alkyl, —(C₁₋₂-alkyl)-(C₃₋₆-cycloalkyl), —(C₁₋₂-alkyl)-heteroaryl and —(C₁₋₂-alkyl)-C≡CH;
   wherein each heterocyclyl is selected from the group consisting of azetidinyl, piperidinyl, tetrahydrofuranyl, tetrahydropyranyl and morpholinyl; and
   wherein each heterocyclyl group is optionally independently substituted with one substituent selected from the group consisting of F, CN, OH, CH₃, —O—CH₃; and
   wherein each heteroaryl is selected from the group consisting of isoxazolyl, thiazolyl and thiadiazolyl; and
   wherein each alkyl, cycloalkyl, heterocyclyl, or heteroaryl group is optionally independently substituted with one or more F, CN, CH₃, —OCH₃, —C(═O)—CH₃ and —C(═O)-cyclopropyl;

or a salt thereof, preferably a pharmaceutically acceptable salt thereof.

Another preferred subgroup of compounds of formula (I) concerns compounds of formula (I.1), wherein

R¹ is selected from the group consisting of cyclopropyl, heterocyclyl and —O—R²;
wherein R² is selected from the group consisting of C₁₋₄-alkyl, —CH₂—(C₃₋₄-cycloalkyl), —CH₂-heteroaryl and —CH₂—CH₂—C≡CH;
   wherein each heteroaryl is selected from the group consisting of isoxazolyl, thiazolyl and thiadiazolyl; and
   wherein each alkyl, cycloalkyl, aryl or heteroaryl group is optionally independently substituted with one or more F, CN and —OCH₃.
   wherein each heterocyclyl is selected from the group consisting of azetidinyl, piperidinyl, tetrahydrofuranyl, tetrahydropyranyl and morpholinyl; and
   wherein each heterocyclyl group is optionally independently substituted with one substituent selected from the group consisting of F, CN, OH, CH₃, —O—CH₃;

or a salt thereof, preferably a pharmaceutically acceptable salt thereof.

Preferred AOC3 inhibitor compounds of the formula (I) are selected from the group G1.2 consisting of compounds (1) to (38) set forth in Table 2 or a pharmaceutically acceptable salts thereof.

TABLE 2

| # | Structure | Chemical Name |
|---|---|---|
| 01 | 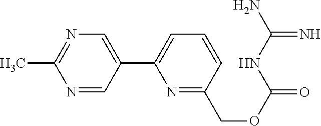 [SMILES: Cc1ncc(-c2cccc(COC(=O)NC(=N)N)n2)cn1] | [6-(2-methylpyrimidin-5-yl)-2- pyridyl]methyl N- carbamimidoylcarbamate |
| 02 | 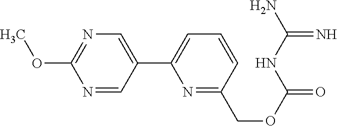 [SMILES: COc1ncc(-c2cccc(COC(=O)NC(=N)N)n2)cn1] | [6-(2-methoxypyrimidin-5-yl)-2- pyridyl]methyl N- carbamimidoylcarbamate |
| 03 | 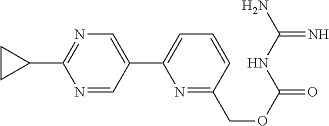 [SMILES: N=C(N)NC(=O)OCc1cccc(-c2cnc(C3CC3)nc2)n1] | [6-(2-cyclopropylpyrimidin-5-yl)-2- pyridyl]methyl N-carbamimidoylcarbamate |
| 04 | 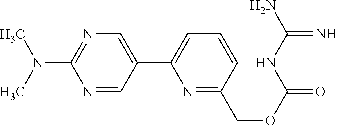 [SMILES: CN(C)c1ncc(-c2cccc(COC(=O)NC(=N)N)n2)cn1] | {6-[2-(dimethylamino)pyrimidin-5-yl]-2- pyridyl}methyl N- carbamimidoylcarbamate |
| 05 | 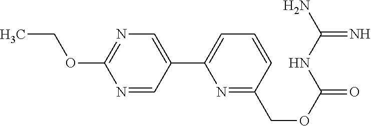 [SMILES: CCOc1ncc(-c2cccc(COC(=O)NC(=N)N)n2)cn1] | [6-(2-ethoxypyrimidin-5-yl)-2- pyridyl]methyl N- carbamimidoylcarbamate |
| 06 | 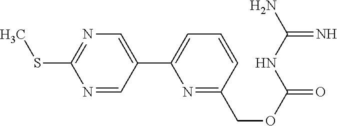 [SMILES: CSc1ncc(-c2cccc(COC(=O)NC(=N)N)n2)cn1] | {6-[2-(methylsulfanyl)pyrimidin-5-yl]-2- pyridyl}methyl N- carbamimidoylcarbamate |
| 07 | 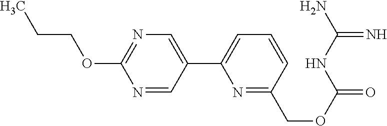 [SMILES: CCCOc1ncc(-c2cccc(COC(=O)NC(=N)N)n2)cn1] | [6-(2-propoxypyrimidin-5-yl)-2-pyridyl]- methyl N-carbamimidoylcarbamate |
| 08 | 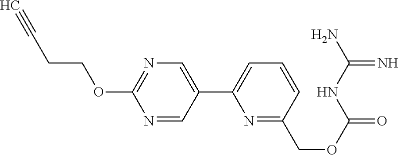 [SMILES: C#CCCOc1ncc(-c2cccc(COC(=O)NC(=N)N)n2)cn1] | [6-(2-but-3-ynoxypyrimidin-5-yl)-2- pyridyl]methyl N-carbamimidoylcarbamate |
| 09 | 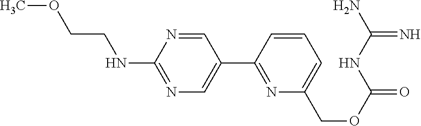 [SMILES: COCCNc1ncc(-c2cccc(COC(=O)NC(=N)N)n2)cn1] | (6-{2-[(2- methoxyethyl)amino]pyrimidin-5-yl}-2- pyridyl)methyl N- carbamimidoylcarbamate |
| 10 | 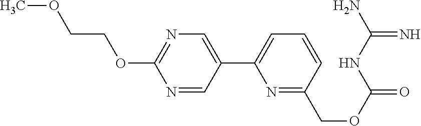 [SMILES: COCCOc1ncc(-c2cccc(COC(=O)NC(=N)N)n2)cn1] | {6-[2-(2-methoxyethoxy)pyrimidin-5-yl]- 2-pyridyl}methyl N- carbamimidoylcarbamate |
| 11 | 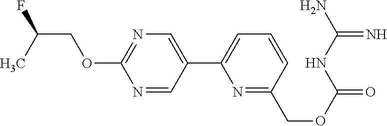 [SMILES: C[C@@H](F)COc1ncc(-c2cccc(COC(=O)NC(=N)N)n2)cn1] | (6-{2-[(2R)-2-fluoropropoxy]pyrimidin- 5-yl}-2-pyridyl)methyl N- carbamimidoylcarbamate |
| 12 | 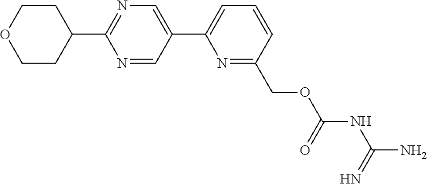 [SMILES: N=C(N)NC(=O)OCc1cccc(-c2cnc(C3CCOCC3)nc2)n1] | {6-[2-(oxan-4-yl)pyrimidin-5-yl]-2- pyridyl}methyl N- carbamimidoylcarbamate |
| 13 | 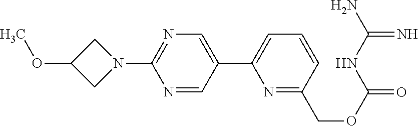 [SMILES: COC1CN(c2ncc(-c3cccc(COC(=O)NC(=N)N)n3)cn2)C1] | {6-[2-(3-methoxyazetidin-1- yl)pyrimidin-5-yl]-2-pyridyl}methyl N- carbamimidoylcarbamate |
| 14 | 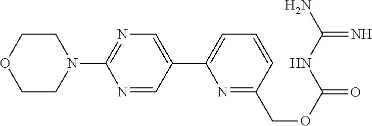 [SMILES: N=C(N)NC(=O)OCc1cccc(-c2cnc(N3CCOCC3)nc2)n1] | {6-[2-(morpholin-4-yl)pyrimidin-5-yl]-2- pyridyl}methyl N- carbamimidoylcarbamate |
| 15 | 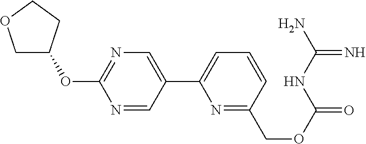 [SMILES: N=C(N)NC(=O)OCc1cccc(-c2cnc(O[C@H]3CCOC3)nc2)n1] | (6-{2-[(3S)-oxolan-3-yloxy]pyrimidin-5- yl}-2-pyridyl)methyl N- carbamimidoylcarbamate |
| 16 | 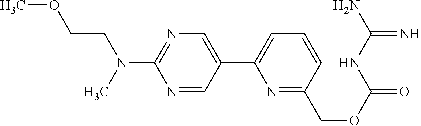 [SMILES: COCCN(C)c1ncc(-c2cccc(COC(=O)NC(=N)N)n2)cn1] | (6-{2-[(2-methoxyethyl)- (methyl)amino]pyrimidin-5-yl}-2- pyridyl)methyl N-carbamimidoylcarbamate |
| 17 | 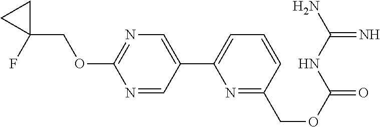 [SMILES: N=C(N)NC(=O)OCc1cccc(-c2cnc(OCC3(F)CC3)nc2)n1] | [6-[2-[(1-fluorocyclopropyl)methoxy]- pyrimidin-5-yl]-2-pyridyl]methyl N- carbamimidoylcarbamate |
| 18 | 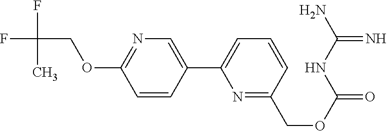 [SMILES: CC(F)(F)COc1ccc(-c2cccc(COC(=O)NC(=N)N)n2)cn1] | [6-[2-(2,2-difluoropropoxy)pyridin-5-yl]-2- pyridyl]methyl N-carbamimidoylcarbamate |
| 19 | 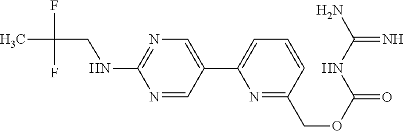 [SMILES: CC(F)(F)CNc1ncc(-c2cccc(COC(=O)NC(=N)N)n2)cn1] | (6-{2-[(2,2-difluoropropyl)amino]pyrimidin- 5-yl}-2-pyridyl)methyl N- carbamimidoylcarbamate |
| 20 | 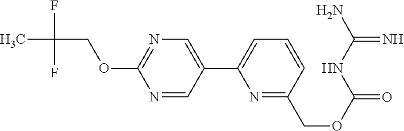 [SMILES: CC(F)(F)COc1ncc(-c2cccc(COC(=O)NC(=N)N)n2)cn1] | [6-[2-(2,2-difluoropropoxy)pyrimidin-5-yl]- 2-pyridyl]methyl N- carbamimidoylcarbamate |
| 21 | 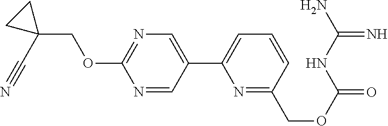 [SMILES: N#CC1(COc2ncc(-c3cccc(COC(=O)NC(=N)N)n3)cn2)CC1] | (6-{2-[(1-cyanocyclopropyl)methoxy]- pyrimidin-5-yl}-2-pyridyl)methyl N- carbamimidoylcarbamate |
| 22 | 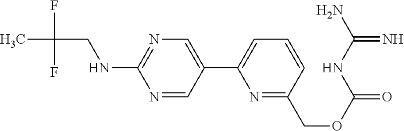 [SMILES: CC(F)(F)CNc1ncc(-c2cccc(COC(=O)NC(=N)N)n2)cn1] | (6-{2-[(2,2-difluoropropyl)amino]pyrimidin- 5-yl}-2-pyridyl)methyl N- carbamimidoylcarbamate |
| 23 | 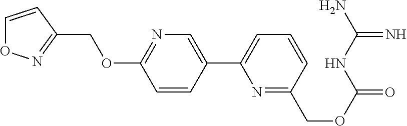 [SMILES: N=C(N)NC(=O)OCc1cccc(-c2ccc(OCc3ccon3)nc2)n1] | (6-{2-[(1,2-oxazol-3-yl)methoxy]pyrimidin- 5-yl}-2-pyridyl)methyl N- carbamimidoylcarbamate |
| 24 | 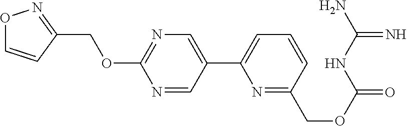 [SMILES: N=C(N)NC(=O)OCc1cccc(-c2cnc(OCc3ccon3)nc2)n1] | [6-[2-(isoxazol-3-ylmethoxy)pyrimidin-5- yl]-2-pyridyl]methyl N- carbamimidoylcarbamate |
| 25 | 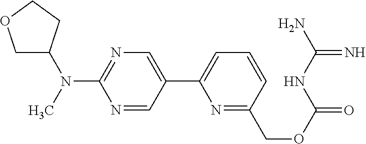 [SMILES: CN(c1ncc(-c2cccc(COC(=O)NC(=N)N)n2)cn1)C1CCOC1] | (6-{2-[methyl(oxolan-3-yl)amino]pyrimidin- 5-yl}-2-pyridyl)methyl N- carbamimidoylcarbamate |
| 26 | 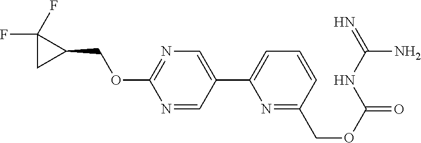 [SMILES: N=C(N)NC(=O)OCc1cccc(-c2cnc(OC[C@H]3CC3(F)F)nc2)n1] | [6-[2-[[(1R)-2,2- difluorocyclopropyl]methoxy]- pyrimidin-5-yl]-2-pyridyl]methyl N- carbamimidoylcarbamate |
| 27 | 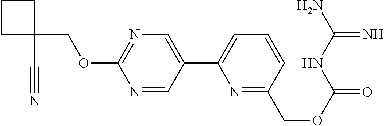 [SMILES: N#CC1(COc2ncc(-c3cccc(COC(=O)NC(=N)N)n3)cn2)CCC1] | (6-{2-[(1- cyanocyclobutyl)methoxy]pyrimidin-5-yl}- 2-pyridyl)methyl N- carbamimidoylcarbamate |
| 28 | 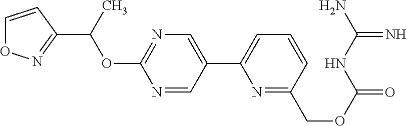 [SMILES: CC(Oc1ncc(-c2cccc(COC(=O)NC(=N)N)n2)cn1)c1ccon1] | (6-{2-[1-(1,2-oxazol-3-yl)ethoxy]pyrimidin- 5-yl}-2-pyridyl)methyl N- carbamimidoylcarbamate |
| 29 | 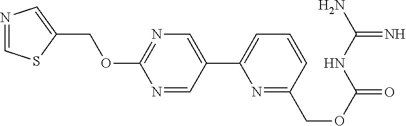 [SMILES: N=C(N)NC(=O)OCc1cccc(-c2cnc(OCc3cncs3)nc2)n1] | (6-{2-[(1,3-thiazol-5-yl)methoxy]pyrimidin- 5-yl}-2-pyridyl)methyl N- carbamimidoylcarbamate |
| 30 | 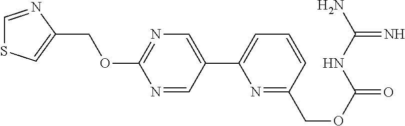 [SMILES: N=C(N)NC(=O)OCc1cccc(-c2cnc(OCc3cscn3)nc2)n1] | (6-{2-[(1,3-thiazol-4-yl)methoxy]pyrimidin- 5-yl}-2-pyridyl)methyl N- carbamimidoylcarbamate |
| 31 | 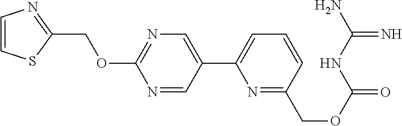 [SMILES: N=C(N)NC(=O)OCc1cccc(-c2cnc(OCc3nccs3)nc2)n1] | (6-{2-[(1,3-thiazol-2-yl)methoxy]pyrimidin- 5-yl}-2-pyridyl)methyl N- carbamimidoylcarbamate |
| 32 | 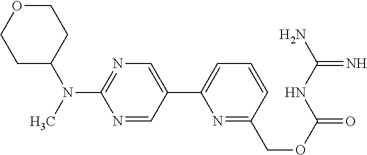 [SMILES: CN(c1ncc(-c2cccc(COC(=O)NC(=N)N)n2)cn1)C1CCOCC1] | (6-{2-[methyl(oxan-4-yl)amino]pyrimidin-5- yl}-2-pyridyl)methyl N- carbamimidoylcarbamate |
| 33 | 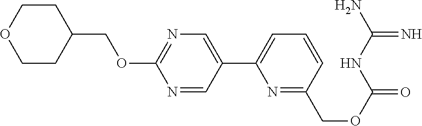 [SMILES: N=C(N)NC(=O)OCc1cccc(-c2cnc(OCC3CCOCC3)nc2)n1] | (6-{2-[(oxan-4-yl)methoxy]pyrimidin-5-yl}- 2-pyridyl)methyl N- carbamimidoylcarbamate |
| 34 | 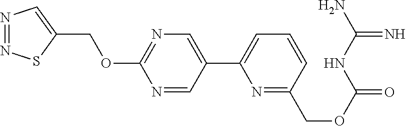 [SMILES: N=C(N)NC(=O)OCc1cccc(-c2cnc(OCc3cnns3)nc2)n1] | (6-{2-[(1,2,3-thiadiazol-5- yl)methoxy]pyrimidin-5-yl}-2-pyridyl)methyl N-carbamimidoylcarbamate |
| 35 | 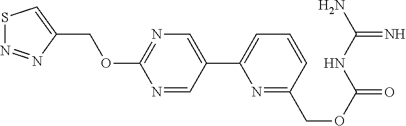 [SMILES: N=C(N)NC(=O)OCc1cccc(-c2cnc(OCc3csnn3)nc2)n1] | (6-{2-[(1,2,3-thiadiazol-4- yl)methoxy]pyrimidin-5-yl}-2-pyridyl)methyl N-carbamimidoylcarbamate |
| 36 | 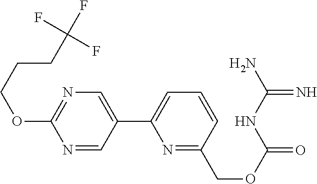 [SMILES: N=C(N)NC(=O)OCc1cccc(-c2cnc(OCCCC(F)(F)F)nc2)n1] | [6-[2-(4,4,4-trifluorobutoxy)pyrimidin-5-yl]- 2-pyridyl]methyl N- carbamimidoylcarbamate |
| 37 | 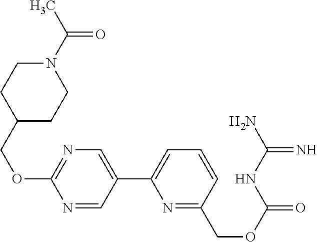 [SMILES: CC(=O)N1CCC(COc2ncc(-c3cccc(COC(=O)NC(=N)N)n3)cn2)CC1] | (6-{2-[(1-acetylpiperidin-4- yl)methoxy]pyrimidin-5-yl}-2-pyridyl)methyl N-carbamimidoylcarbamate |
| 38 | 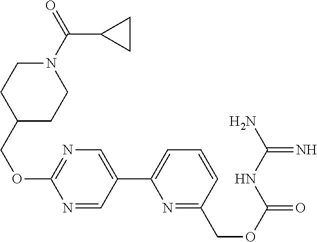 [SMILES: N=C(N)NC(=O)OCc1cccc(-c2cnc(OCC3CCN(C(=O)C4CC4)CC3)nc2)n1] | (6-{2-[(1-cyclopropanecarbonylpiperidin-4- yl)methoxy]pyrimidin-5-yl}-2-pyridyl)methyl N-carbamimidoylcarbamate |

More preferred AOC3 inhibitor compounds of the formula (I) are selected from the group G1.3 consisting of compounds No. 3, 12, 20, 24, 26 and 36 figuring below or a pharmaceutically acceptable salt thereof:

| # | Structure |
|---|---|
| 3 | 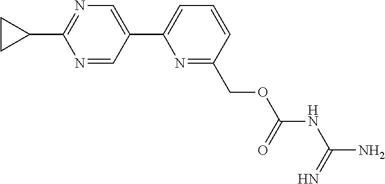 [SMILES: N=C(N)NC(=O)OCc1cccc(-c2cnc(C3CC3)nc2)n1] |
| 12 | 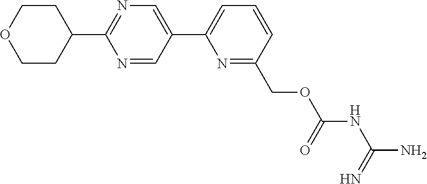 [SMILES: N=C(N)NC(=O)OCc1cccc(-c2cnc(C3CCOCC3)nc2)n1] |
| 20 | 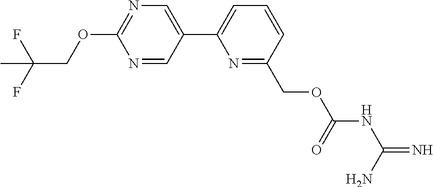 [SMILES: CC(F)(F)COc1ncc(-c2cccc(COC(=O)NC(=N)N)n2)cn1] |
| 24 | 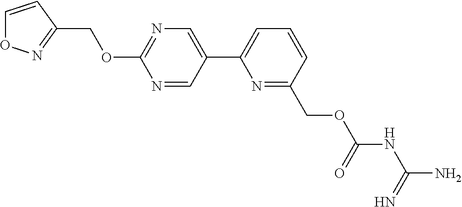 [SMILES: N=C(N)NC(=O)OCc1cccc(-c2cnc(OCc3ccon3)nc2)n1] |
| 26 | 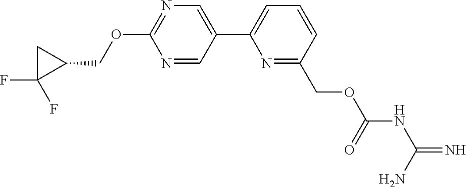 [SMILES: N=C(N)NC(=O)OCc1cccc(-c2cnc(OC[C@H]3CC3(F)F)nc2)n1] |
| 36 | 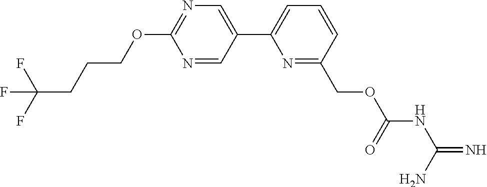 [SMILES: N=C(N)NC(=O)OCc1cccc(-c2cnc(OCCCC(F)(F)F)nc2)n1] |

The AOC3 inhibitors according to this invention are potent inhibitors of the human AOC3 enzyme and have much advantageous pharmacological and safety properties. These compounds are very weak inhibitors of other family members, such as monoamine oxidase A, monoamine oxidase B, diamine oxidase, lysyl oxidase, and lysyl-like amine oxidases LOX1-4. Preferred AOC3 inhibitors, in particular those selected from the above group G1.2, have a high inhibitory potency against human AOC3 and a low inhibitory activity against human diamine oxidase.

Preferably AOC3 inhibitor compounds of the formula (I) are selected from the group G1.3 consisting of compounds (3), (12), (20), (24), (26) and (36) of Table 2, or pharmaceutically acceptable salts thereof.

According to this invention, it is to be understood that the definitions of the above listed AOC3 inhibitors of the formula (I) also comprise their pharmaceutically acceptable salts, solvates and polymorphic forms thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, and commensurate with a reasonable benefit/risk ratio.

As used herein, “pharmaceutically acceptable salts” refer to derivatives of the disclosed compounds wherein the parent compound is modified by making acid or base salts thereof. According to one embodiment, the pharmaceutical acceptable salt is an acid addition salt. According to another embodiment, the pharmaceutical acceptable salt is a base salt.

Examples of pharmaceutically acceptable salts include, but are not limited to, mineral or organic acid salts of basic residues such as amines; alkali or organic salts of acidic residues such as carboxylic acids; and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a sufficient amount of the appropriate base or acid in water or in an organic diluent like ether, ethyl acetate, ethanol, isopropanol, or acetonitrile, or a mixture thereof.

The aspects according to the present invention, in particular the pharmaceutical compositions, methods and uses, refer to an SGLT2 inhibitor. In the following preferred SGLT2 inhibitors according to this invention are described.

Preferably the SGLT2 inhibitor is selected from the group G2 consisting of empagliflozin, dapagliflozin, canagliflozin, ipragliflozin, tofogliflozin, luseogliflozin, atigliflozin, remogliflozin, sergliflozin, ertugliflozin and sotagliflozin.

More preferably the SGLT2 inhibitor is selected from the group G2.1 consisting of empagliflozin, dapagliflozin, canagliflozin, ipragliflozin, tofogliflozin, luseogliflozin.

A preferred SGLT2 inhibitor is empagliflozin which has a high selectivity SGLT2 versus SGLT1 (Grempler et al., Diabetes, Obesity and Metabolism, 2012, 14, 83-90) and which in patients with type 2 diabetes mellitus at high risk for cardiovascular events has significantly lower rates of the primary composite cardiovascular outcome and of death from any cause (Zinman, et al., N. Engl. J. Med. 2015, 373, 2117-2128).

The term “empagliflozin” as employed herein refers to empagliflozin, including hydrates and solvates thereof, and crystalline forms thereof. The compound and methods of its synthesis are described in WO 2005/092877, WO 2006/120208, WO 2011/039108 for example. A crystalline form is described in the patent applications WO 2006/117359, WO 2011/039107 for example.

The term “dapagliflozin” as employed herein refers to dapagliflozin, including hydrates and solvates thereof, and crystalline forms thereof. The compound and methods of its synthesis are described in WO 03/099836 for example. Preferred hydrates, solvates and crystalline forms are described in the patent applications WO 2008/116179 and WO 2008/002824 for example.

The term “canagliflozin” as employed herein refers to canagliflozin, including hydrates and solvates thereof, and crystalline forms thereof. The compound and methods of its synthesis are described in WO 2005/012326 and WO 2009/035969 for example. Preferred hydrates, solvates and crystalline forms are described in the patent applications WO 2008/069327 for example.

The term “ipragliflozin” as employed herein refers to ipragliflozin, including hydrates and solvates thereof, and crystalline forms thereof. The compound and methods of its synthesis are described in WO 2004/080990 for example.

The term “tofogliflozin” as employed herein refers to tofogliflozin, including hydrates and solvates thereof, and crystalline forms thereof. The compound and methods of its synthesis are described in WO 2006/080421, WO 2007/140191, WO 2009/154276 for example.

The term “luseogliflozin” as employed herein refers to luseogliflozin, including hydrates and solvates thereof, and crystalline forms thereof. The compound and methods of its synthesis are described in WO 2006/073197, WO 2010/119990 for example

The term “atigliflozin” as employed herein refers to atigliflozin, including hydrates and solvates thereof, and crystalline forms thereof. The compound and methods of its synthesis are described in WO 2004/007517 for example.

The term “remogliflozin” as employed herein refers to remogliflozin and prodrugs of remoflozin, in particular sergliflozin etabonate, including hydrates and solvates thereof, and crystalline forms thereof. Methods of its synthesis are described in the patent applications EP 1213296 and EP 1354888 for example.

The term “sergliflozin” as employed herein refers to sergliflozin and prodrugs of sergliflozin, in particular sergliflozin etabonate, including hydrates and solvates thereof, and crystalline forms thereof. Methods for its manufacture are described in the patent applications EP 1344780 and EP 1489089 for example.

The term “ertugliflozin” as employed herein refers to ertugliflozin and including hydrates and solvates thereof, and crystalline forms thereof. Methods for its manufacture are described in the patent applications WO 2010/023594 for example.

The term “sotagliflozin” as employed herein refers to sotagliflozin and including hydrates and solvates thereof, and crystalline forms thereof. Methods for its manufacture are described in the patent applications WO 2008/109591, WO 2008/042688, WO 2009/014970, WO 2010/009197 for example.

For avoidance of any doubt, the disclosure of each of the foregoing documents cited above in connection with the specified SGLT2 inhibitors is specifically incorporated herein by reference in its entirety.

In a first embodiment E1 the pharmaceutical combination or compositions, methods and uses according to this invention preferably relate to a AOC3 inhibitor of formula (I) which is selected from the group G1 consisting of compounds of formula (I)

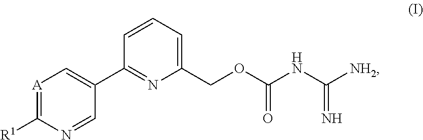

wherein R¹ and A are as defined as hereinbefore, or a pharmaceutically acceptable salt thereof. According to an embodiment the AOC3 inhibitor is selected from the group G1.1 consisting of compounds of formula (I) or a pharmaceutically acceptable salt thereof as defined as hereinbefore. More preferably the AOC3 inhibitor of formula (I) is selected from the group G1.2 consisting of compounds (1) to (38) or a pharmaceutically acceptable salt thereof as defined hereinbefore. Even more preferably the AOC3 inhibitor of formula (I) is selected from the group G1.3 consisting of compounds (3), (12), (20), (24), (26) and (36) or a pharmaceutically acceptable salt thereof as defined hereinbefore.

In the first embodiment E1 the pharmaceutical combination or compositions, methods and uses according to this invention preferably relate to a SGLT2 inhibitor selected from the group G2 consisting of empagliflozin, dapagliflozin, canagliflozin, ipragliflozin, tofogliflozin, luseogliflozin, atigliflozin, remogliflozin, sergliflozin, ertugliflozin and sotagliflozin as defined hereinbefore. Preferably the SGLT2 inhibitor selected from the group G2.1 consisting of empagliflozin, dapagliflozin, canagliflozin, ipragliflozin, tofogliflozin, luseogliflozin. For example the SGLT2 inhibitor is empagliflozin.

According to the first embodiment E1 the SGLT2 inhibitors are preferably selected according to the entries in the Table 3.

TABLE 3

| Embodiment | AOC3 inhibitor | SGLT2 Inhibitor |
|---|---|---|
| E1.1 | selected from | selected from |
| | the group G1 | the group G2 |
| E1.2 | selected from | selected from |
| | the group G1.1 | the group G2 |
| E1.3 | selected from | selected from |
| | the group G1.2 | the group G2 |
| E1.4 | selected from | selected from |
| | the group G1.3 | the group G2 |
| E1.5 | selected from | selected from |
| | the group G1 | the group G2.1 |
| E1.6 | selected from | selected from |
| | the group G1.1 | the group G2.1 |
| E1.7 | selected from | selected from |
| | the group G1.2 | the group G2.1 |
| E1.8 | selected from | selected from |
| | the group G1.3 | the group G2.1 |
| E1.9 | selected from | empagliflozin |
| | the group G1 | |
| E1.10 | selected from | empagliflozin |
| | the group G1.1 | |
| E1.11 | selected from | empagliflozin |
| | the group G1.2 | |
| E1.12 | selected from | empagliflozin |
| | the group G1.3 | |
| E1.13 | compound (2) | selected from |
| | | the group G2 |
| E1.14 | compound (12) | selected from |
| | | the group G2 |
| E1.15 | compound (20) | selected from |
| | | the group G2 |
| E1.16 | compound (24) | selected from |
| | | the group G2 |
| E1.17 | compound (26) | selected from |
| | | the group G2 |
| E1.18 | compound (36) | selected from |
| | | the group G2 |
| E1.19 | compound (2) | selected from |
| | | the group G2.1 |
| E1.20 | compound (12) | selected from |
| | | the group G2.1 |
| E1.21 | compound (20) | selected from |
| | | the group G2.1 |
| E1.22 | compound (24) | selected from |
| | | the group G2.1 |
| E1.23 | compound (26) | selected from |
| | | the group G2.1 |
| E1.24 | compound (36) | selected from |
| | | the group G2.1 |
| E1.25 | compound (2) | empagliflozin |
| E1.26 | compound (12) | empagliflozin |
| E1.27 | compound (20) | empagliflozin |
| E1.28 | compound (24) | empagliflozin |
| E1.29 | compound (26) | empagliflozin |
| E1.30 | compound (36) | empagliflozin |

Among the combinations according to the present invention listed in Table 3, the combinations No. E1.9, E1.10, E1.11 and E1.12, in which the AOC3 inhibitor of formula (I) is a compound of the group G1, G1.1, G1.2 or G1.3, or a stereoisomer, pharmaceutically acceptable salt, solvate and polymorphic form thereof, and the SGLT2-inhibitor is empagliflozin or a pharmaceutically acceptable salt thereof, are preferred.

In particular, the combinations No. E1.25. E1.26, E1.27. E1.28, E1.29 and E1.30 are preferred, wherein the AOC3 inhibitor of formula (I) is one of the compounds (2), (12), (20), (24), (26) and (36) of Table 2, or a pharmaceutically acceptable salt thereof and the SGLT2-inhibitor is empagliflozin.

According to an embodiment of this aspect the invention relates to a method for preventing, slowing the progression of, delaying or treating of one or more fibrotic diseases, metabolic diseases, inflammatory diseases, ocular diseases, neuroinflammatory diseases, vascular diseases or cancers in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

According to an embodiment of this aspect the invention relates to a method for preventing, slowing the progression of, delaying or treating of a fibrotic disease selected from the group consisting of cystic fibrosis, interstitial lung disease, including idiopathic pulmonary fibrosis, liver fibrosis including non-alcoholic steatohepatitis (NASH), alcohol induced fatty liver, alcohol induced liver fibrosis, toxic fatty liver and cirrhosis of the liver, kidney fibrosis, scleroderma, radiation-induced fibrosis and other diseases where excessive fibrosis contributes to disease pathology in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

According to an embodiment of this aspect the invention relates to a method for preventing, slowing the progression of, delaying or treating of a metabolic disease selected from the group consisting of pre-diabetes mellitus, type 1 diabetes mellitus, type 2 diabetes mellitus, complications associated with diabetes mellitus, overweight, obesity, impaired glucose tolerance (IGT), impaired fasting blood glucose (IFG), hyperglycemia, postprandial hyperglycemia, insulin resistance, fatty liver, including non-alcoholic fatty liver disease (NAFLD), overweight, obesity, metabolic syndrome in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

Complications associated with diabetes mellitus include cataracts and micro- and macrovascular diseases, such as diabetic nephropathy, glomerulosclerosis, diabetic retinopathy, choroidal neovascularisation, non-alcoholic fatty liver (NAFL) disease, non-alcoholic steatohepatitis (NASH), diabetic neuropathy, diabetic pain, tissue ischaemia, diabetic foot, diabetic ulcer, arteriosclerosis, myocardial infarction, accute coronary syndrome, unstable angina pectoris, stable angina pectoris, stroke, peripheral arterial occlusive disease, cardiomyopathy, heart failure, cardiovascular death, heart rhythm disorders and vascular restenosis.

According to another embodiment related to the treatment of a metabolic disease, the invention provides a method for improving glycemic control and/or for reducing of fasting plasma glucose, of postprandial plasma glucose and/or of glycosylated hemoglobin HbA1c in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

By the administration of a pharmaceutical combination or composition according to the present invention, an abnormal accumulation of ectopic fat, in particular of the liver, may be reduced or inhibited. Therefore, according to another embodiment of the present invention, there is provided a method for preventing, slowing, delaying or treating a metabolic disease selected from the group consisting of diseases or conditions attributed to an abnormal accumulation of ectopic fat, in particular of the liver, in a patient in need thereof characterized in that an inhibitor of the human AOC3 enzyme of formula (I) as defined hereinbefore and hereinafter and an SGLT2-inhibitor as defined hereinbefore and hereinafter are administered to the patient. Diseases or conditions which are attributed to an abnormal accumulation of liver fat are particularly selected from the group consisting of general fatty liver, non-alcoholic fatty liver (NAFL), non-alcoholic steatohepatitis (NASH), hyperalimentation-induced fatty liver, diabetic fatty liver, alcoholic-induced fatty liver and toxic fatty liver.

According to another embodiment of this aspect the invention relates to a method for preventing, slowing the progression of, delaying or treating a treating of an inflammation disease selected from the group consisting of arthritis (including juvenile rheumatoid arthritis), Crohn's disease, ulcerative colitis, inflammatory bowel diseases (e.g. irritable bowel disease), psoriasis, asthma (e.g. eosinophilic asthma, severe asthma, virally exacerbated asthma), pulmonary inflammation, chronic pulmonary obstructive disease (COPD), bronchiectasis, skin inflammation, ocular disease, contact dermatitis, liver inflammation, liver autoimmune diseases, autoimmune hepatitis, primary biliary cirrhosis, sclerosing cholangitis, autoimmune cholangitis, alcoholic liver disease, artherosclerosis, chronic heart failure, congestive heart failure, ischemic diseases, stroke and complications thereof, myocardial infarction and complications thereof, inflammatory cell destruction following stroke, synovitis, systemic inflammatory sepsis, inflammation due to diabetes, lung inflammation associated with cystic fibrosis, other bacteria-induced lung diseases such as sepsis, acute respiratory distress syndrome (ARDS), acute lung injury (ALI), transfusion induced lung injury (TRALI) in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

According to another embodiment of this aspect the invention relates to a method for preventing, slowing the progression of, delaying or treating an ocular disease, including macular degeneration, including diabetic macular edema, uveitis and retinopathy, including diabetic retinopathy, in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

According to another embodiment of this aspect the invention relates to a method for preventing, slowing the progression of, delaying or treating of a neuroinflammatory disorder selected from the group consisting of stroke, Parkinson's disease, Alzheimer's disease, vascular dementia, multiple sclerosis, chronic multiple sclerosis in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

According to another embodiment of this aspect the invention relates to a method for preventing, slowing the progression of, delaying or treating a cancer selected from the group consisting of lung cancer, breast cancer, colorectal cancer, anal cancer, pancreatic cancer, prostate cancer, ovarian carcinoma, liver and bile duct carcinoma, esophageal carcinoma, non-Hodgkin's lymphoma, bladder carcinoma, carcinoma of the uterus, glioma, glioblastoma, medullablastoma, and other tumors of the brain kidney cancer, cancer of the head and neck, cancer of the stomach, multiple myeloma, testicular cancer, germ cell tumor, neuroendocrine tumor, cervical cancer, carcinoids of the gastrointestinal tract, breast, and other organs; signet ring cell carcinoma, mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma or a leiomysarcoma in a patient in need thereof characterized in that an AOC3 inhibitor of formula (I) as defined hereinbefore and hereinafter and an SGLT2-inhibitor as defined hereinbefore and hereinafter are administered, for example in combination or alternation, to the patient.

The combination of an AOC3 inhibitor of formula (I) as defined hereinbefore and hereinafter and an SGLT2-inhibitor according to this invention significantly improves various aspects of diseases mentioned hereinbefore and hereinafter, in particular of diabetes and diabetes related complications. According to one aspect treatment of patients with a combination according to the present invention will normalize the hyperglycemia which is the main driver of microvascular and macrovascular complications, dyslipidemia and beta cell failure. According to another aspect treatment of patients with a combination according to the present invention will decrease tissue inflammation and leukocyte recruitment and decrease the pro-inflammatory situation associated with metabolic syndrome and diabetes complications. A treatment employing a combination according to the present invention will decrease the diabetic root cause as well as the pro-inflammatory driver of symptoms and complications associated with diabetes. Furthermore the combination could lead to acceleration of disease resolution or to improvements on symptoms and complications not met by the single mode of action. This could include but is not limited to effects on parameters of metabolic syndrome like insulin sensitivity, effects on weight loss, dyslipidemia, parameters of liver disease, diabetic retinopathy and cardiovascular effects. In addition effects could be improvements on pain, wound healing and improvements on peripheral neurosenstivity, especially in the context of diabetes.

When this invention refers to patients requiring treatment or prevention, it relates primarily to treatment and prevention in humans, but the pharmaceutical composition may also be used accordingly in veterinary medicine in mammals. In the scope of this invention adult patients are preferably humans of the age of 18 years or older. Also in the scope of this invention, patients are adolescent humans, i.e. humans of age 10 to 17 years, preferably of age 13 to 17 years.

The pharmaceutical combinations or compositions, methods and uses according to this invention are advantageously applicable in those patients who show one, two or more of the following conditions:

(a) pre-diabetes

(b) hyperinsulinemia

(c) type 2 diabetes mellitus

(d) type 1 diabetes mellitus

(d) overweight

(e) obesity.

It will be appreciated that the amount of the pharmaceutical composition according to this invention to be administered to the patient and required for use in treatment or prophylaxis according to the present invention will vary with the route of administration, the nature and severity of the condition for which treatment or prophylaxis is required, the age, weight and condition of the patient, concomitant medication and will be ultimately at the discretion of the attendant physician.

In the following preferred ranges of the amount of the AOC3 inhibitor of formula (I) as defined hereinbefore and hereinafter and the SGLT2 inhibitor as defined hereinbefore and hereinafter to be employed in the pharmaceutical combination or composition and the methods and uses according to this invention are described. These ranges refer to the amounts to be administered per day with respect to an adult patient, in particular to a human being, for example of approximately 70 kg body weight, and can be adapted accordingly with regard to an administration 2, 3, 4 or more times daily and with regard to other routes of administration and with regard to the age of the patient. The ranges of the dosage and amounts are calculated for the individual active moiety.

Within the scope of the present invention, the pharmaceutical composition is preferably administered orally. Other forms of administration are possible and described hereinafter. Preferably the one or more dosage forms comprising the AOC3 inhibitor and the SGLT2 inhibitor is a solid pharmaceutical dosage form for oral administration.

The pharmaceutical combinations or compositions, in another embodiment, should provide a dosage of from about 0.001 mg to about 10 mg of the AOC3 inhibitor per kilogram of body weight per day. Each dosage unit may conveniently contain 0.1 to 1000 mg of the active substance, preferably it contains between 0.5 to 500 mg of the AOC3 inhibitor.

Preferably the administration of said amounts is once, twice or three times daily. Suitable formulations for an AOC3 inhibitor of formula (I) as defined hereinbefore and hereinafter may be those formulations disclosed in the application WO 2017/194453, the disclosure of which is incorporated herein in its entirety.

In general, the amount of the SGLT2 inhibitor in the pharmaceutical composition and methods according to this invention is preferably the amount usually recommended for a monotherapy using said SGLT2 inhibitor.

The preferred dosage range of the SGLT2 inhibitor is in the range from 0.5 mg to 200 mg, even more preferably from 1 to 100 mg, most preferably from 1 to 50 mg per day. The oral administration is preferred. Therefore, a pharmaceutical composition may comprise the hereinbefore mentioned amounts, in particular from 1 to 50 mg or 1 to 25 mg. Particular dosage strengths (e.g. per tablet or capsule) are for example 1, 2.5, 5, 7.5, 10, 12.5, 15, 20, 25 or 50 mg, in particular of empagliflozin, or of dapagliflozin. Examples of amounts or dosage strengths per day of empaglfilozin are 1 mg, 2.5 mg, 5 mg, 10 mg and 25 mg in the combinations, compositions, methods or uses according to this invention. The application of the active ingredient may occur one or two times a day. Suitable formulations for empagliflozin may be those formulations disclosed in the application WO 2010/092126, the disclosure of which is incorporated herein in its entirety.

The amount of the AOC3 inhibitor according to the formula (I) and the SGLT2 inhibitor in the pharmaceutical combinations or compositions and in the methods and uses according to this invention correspond to the respective dosage ranges as provided hereinbefore. For example, preferred dosage ranges in a pharmaceutical composition and in methods and uses according to this invention are an amount from about 0.1 mg to about 1000 mg or from about 0.5 mg to about 500 mg of the AOC3 inhibitor according to the formula (I), in particular of compound (2), (12), (20), (24), (26) or (36), and an amount of 1 to 50 mg (in particular 1 to 25 mg) of an SGLT2 inhibitor according to the formula (I), in particular of empagliflozin, e.g. in an amount of 10 mg or 25 mg. An oral administration once or twice daily is preferred, most preferably once daily.

In the methods and uses according to the present invention the AOC3 inhibitor according to the formula (I) and the SGLT2 inhibitor are administered in combination or alternation. The term “administration in combination” means that the active ingredients are administered at the same time, i.e. simultaneously, or essentially at the same time. The term “administration in alternation” means that at first one active ingredient is administered and after a period of time the other one active ingredients is administered. The period of time may be in the range from 30 min to 12 hours. The administration which is in combination or in alternation may be once, twice, three times or four times daily, preferably once or twice daily, most preferably once daily.

With regard to the administration of the AOC3 inhibitor according to the formula (I) and the SGLT2 inhibitor, the two active ingredients may be present in one single dosage form, for example in one tablet or capsule, or the active ingredients may be present in separate dosage forms, for example in two different or identical dosage forms.

With regard to their administration in alternation, the active ingredients are present in a separate dosage form, for example in two different or identical dosage forms.

Therefore, the pharmaceutical composition according to this invention may be present as a single dosage form which comprises the AOC3 inhibitor according to the formula (I) and the SGLT2 inhibitor. Alternatively the pharmaceutical composition according to this invention may be present as two separate dosage forms wherein one dosage form comprises the AOC3 inhibitor according to the formula (I) and the other dosage form comprises the SGLT2 inhibitor.

The case may arise in which one active ingredient has to be administered more often, for example twice per day, than the other active ingredients, which for example needs administration once daily. Therefore the term “administration in combination or alternation” also includes an administration scheme in which first all active ingredients are administered in combination or alternation and after a period of time only one active ingredient is administered again or vice versa.

Therefore, the present invention also includes pharmaceutical compositions which are present in separate dosage forms wherein one dosage form comprises the AOC3 inhibitor according to the formula (I) and the SGLT2 inhibitor and the other dosage form comprises the AOC3 inhibitor according to the formula (I) only.

A pharmaceutical composition which is present as a separate or multiple dosage form, preferably as a kit of parts, is useful in combination therapy to flexibly suit the individual therapeutic needs of the patient.

According to a first embodiment a preferred kit of parts comprises
(a) a first containment containing a dosage form comprising the AOC3 inhibitor according to the formula (I) and at least one pharmaceutically acceptable carrier, and
(b) a further containment containing a dosage form comprising the SGLT2 inhibitor and at least one pharmaceutically acceptable carrier.

A further aspect of the present invention is a manufacture comprising the pharmaceutical composition being present as separate dosage forms according to the present invention and a label or package insert comprising instructions that the separate dosage forms are to be administered in combination or alternation.

According to a first embodiment a manufacture comprises (a) a pharmaceutical composition comprising an AOC3 inhibitor according to the formula (I) according to the present invention and (b) a label or package insert which comprises instructions that the medicament may or is to be administered, for example in combination or alternation, with a medicament comprising an SGLT2 inhibitor according to the present invention.

According to a second embodiment a manufacture comprises (a) a pharmaceutical composition comprising an SGLT2 inhibitor according to the present invention and (b) a label or package insert which comprises instructions that the medicament may or is to be administered, for example in combination or alternation, with a medicament comprising a an AOC3 inhibitor of the formula (I) according to the present invention.

The desired dose of the pharmaceutical composition according to this invention may conveniently be presented in a once daily or as divided dose administered at appropriate intervals, for example as two, three or more doses per day.

The pharmaceutical composition may be formulated for oral, rectal, nasal, topical (including buccal and sublingual), transdermal, vaginal or parenteral (including intramuscular, sub-cutaneous and intravenous) administration in liquid or solid form or in a form suitable for administration by inhalation or insufflation. Oral administration is preferred. The formulations may, where appropriate, be conveniently presented in discrete dosage units and may be prepared by any of the methods well known in the art of pharmacy. All methods include the step of bringing into association the active ingredient with one or more pharmaceutically acceptable carriers, like liquid carriers or finely divided solid carriers or both, and then, if necessary, shaping the product into the desired formulation.

The pharmaceutical composition may be formulated in the form of tablets, granules, fine granules, powders, capsules, caplets, soft capsules, pills, oral solutions, syrups, dry syrups, chewable tablets, troches, effervescent tablets, drops, suspension, fast dissolving tablets, oral fast-dispersing tablets, etc.

The pharmaceutical composition and the dosage forms preferably comprises one or more pharmaceutical acceptable carriers. Preferred carriers must be “acceptable” in the sense of being compatible with the other ingredients of the formulation and not deleterious to the recipient thereof. Examples of pharmaceutically acceptable carriers are known to the one skilled in the art.

Pharmaceutical compositions suitable for oral administration may conveniently be presented as discrete units such as capsules, including soft gelatin capsules, cachets or tablets each containing a predetermined amount of the active ingredient; as a powder or granules; as a solution, a suspension or as an emulsion, for example as syrups, elixirs or self-emulsifying delivery systems (SEDDS). The active ingredients may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The pharmaceutical composition according to the invention may also be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilisation from solution, for constitution with a suitable vehicle, e.g. sterile, pyrogen-free water, before use.

Pharmaceutical compositions suitable for rectal administration wherein the carrier is a solid are most preferably presented as unit dose suppositories. Suitable carriers include cocoa butter and other materials commonly used in the art, and the suppositories may be conveniently formed by admixture of the active compound(s) with the softened or melted carrier(s) followed by chilling and shaping in moulds.

The methods of synthesis for AOC3 inhibitor according to the formula (I) are described WO2017/194453, the disclosures of which are incorporated herein.

The methods of synthesis for SGLT2 inhibitors are described in the scientific literature and/or in published patent documents, particularly in those cited hereinbefore.

With respect to empagliflozin, the methods of synthesis are known to the one skilled in the art. Advantageously, the compounds according to this invention can be prepared using synthetic methods as described in the literature, in particular as described in the WO 2005/092877, WO 2006/120208 and WO 2011/039108, the disclosures of which are incorporated herein. An advantageous crystalline form is described in the international patent application WO 2006/117359 and WO 2011/039107, which hereby is incorporated herein in its entirety.

Any of the above mentioned combinations and methods within the scope of the invention may be tested by animal models known in the art. In the following, in vivo experiments are described which are suitable to evaluate pharmacologically relevant properties of pharmaceutical compositions and methods according to this invention, animal experiments in appropriate species that allow the analysis of diabetes and diabetes derived complications, eye diseases, tissue fibrosis, inflammation or cancer.

Models could in principle include genetic predisposition and treatments like specific diets, surgery, or toxic agents or combinations thereof. Models of diabetes could include but are not limited to genetically induced diabetes like the db/db mouse, KKAy mouse and other mouse strains, the ZDF rat and other rat strains, diet induced diabetes in rats or mice, age induced diabetes, or toxic agent like streptozotocine induced diabetes and combinations thereof. Models of eye disease could include but are not limited to studies of vasculature permeability and angiogenesis like models of oxygen induced retinopathy model in mice, diabetes induced retinopathy, and models of injury induced eye disease like laser induced choroideal neoangiogenesis or retinal vein occlusion model. Models of chronic kidney disease could include the ZSF rat treated with specific diets like the high fat diet. Models of artherosclerosis could include the ApoE mouse and others and the treatment with pro-artherogenic diets. Models of inflammation could include lung inflammation induced by instillation or inhalation of toxic agents like LPS or cigarette smoke, virus- or bacterial preparations, cytokines or others. Tissue inflammation could include the injection or topical application of above reagents. Models of neuroinflammation could include the above treatment as well as transgenic animals positive for mutations of Abeta and/or tau proteins. Models of fibrosis could include but are not limited to models of liver fibrosis induced by diet protocols like the high fat diet, the methionine-choline deficient diet, choline-deficient aminoacid defined diet and diets enriched with cholesterol. Further treatments including liver toxic agents like tetrachloro carbon, thioacetamide, lipopolysaccharide, dextransulfate and others as well as combinations thereof. Genetic strains that develop spontaneous liver fibrosis like the Mdr2 knock out mouse or strains that excert a susceptibility for liver fibrosis like the Nrf2 knock out mouse upon treatment with protocols described above. Finally models include surgery protocols surgery like bile duct ligation for the generation of liver fibrosis.

Other tissue fibrosis models could include lung fibrosis induced by toxic agents like bleomycin or kidney fibrosis including unilateral ureteral obstruction (UUO) surgery.

Pharmacological Examples

The following example shows the beneficial effect on glycemic control, body weight, body composition and anti-inflammatory and anti-fibrotic effects of the combination and pharmaceutical compositions according to the present invention.

### Example 1: Animal In Vivo Experiment

### Animals Treatment

The C57BL/6 mice are maintained under controlled conditions of temperature (23±2° C.), humidity (45±10%), lighting (12-hour artificial light and dark cycles) and air exchange. Induction of a diabetes dependent NASH phenotype (Teruo Jojima et al., Diabetol Metab Syndr (2016) 8:45) is achieved by a single subcutaneous injection of streptozotocin (200 μg, Sigma-Aldrich, USA) solution 2 days after birth and feeding with high fat diet (HFD, 57 kcal % fat,cat #: HFD32, CLEA Japan, Japan) and drinking water ad libitum after 4 weeks of age. This mouse model progresses from NAFLD to NASH by 8 weeks of age.

Vehicle, the compound (23) (in the form of is hydrochloride salt) and empagliflozin are administered orally to mice in a volume of 10 mL/kg body weight at the end of the light cycle starting from week 7 to week 10. Dosing groups include 10 male animals. For example the dosing of the compound (23) (as HCl salt) is 2 mg/kg and 10 mg/kg for empagliflozin once daily. The combination includes compound (23) (as HCl salt) (2 mg/kg) and empagliflozin (10 mg/kg).

Body Weight & Food Intake

Body weight and food and water intake data are recorded. In the case of body weight analysis, Day 1 body weight (i.e. the weight immediately before the first drug treatment) is the covariate. In the case of the food and water intake analysis, the covariate is the average daily intake during the baseline phase of the study.

Measurement of Plasma Biochemistry

For plasma biochemistry, blood is collected by heart puncture with an anticoagulant (Novo-Heparin; Mochida Pharmaceutical, Japan)-coated syringe. Plasma is generated by centrifugation at 1,000×g for 15 minutes at 4° C. The plasma samples are frozen immediately and thawed just before analysis. Blood levels of alanine aminotransferase (ALT), triglycerides (TG), free fatty acids (FFA), and glycated albumin (GA) are measured with an auto-analyzer (JEOL Ltd., Tokyo, Japan). Further plasma parameters are assayed by commercial kits e.g. glucose (Thermo Electron Corp., PA, USA) and insulin (Mercodia, Uppsala, Sweden). Blood (collected in an EDTA tube and frozen immediately) is assayed for HbA1c by a direct enzymatic assay (Diazyme, CA, USA).

Measurement of Liver TG

Liver total lipid-extracts are obtained by Folch's method (Folch J. et al, J Biol Chem 1957; 226:497). The liver samples are homogenized in chloroform-methanol (2:1, v/v) and incubated for 12 h at room temperature. After washing with chloroform-methanol-water (8:4:3, v/v/v), the samples are evaporated to dryness and afterwards dissolved in isopropanol. Total TG content are measured by Triglyceride E-test (Wako Pure Chemical Industries).

Histopathological and Immunohistochemical Analyses

Tissue sections are cut from paraffin blocks of liver samples prefixed in Bouin's solution and stained with Lillie-Mayer's Hematoxylin (Muto Pure Chemicals, Japan) and eosin solution SR_MNP036-1208-6 8/15 (Wako Pure Chemical Industries). NAFLD Activity score (NAS) is calculated according to Kleiner criteria (Kleiner D E et al. Hepatology 2005; 41:1313). Collagen deposition is visualized by staining of Bouin's fixed liver sections with picro-Sirius red solution (Waldeck GmbH & Co., Germany).

Gene Expression Analysis

Liver samples from animal studies are preserved in RNAlater™ (Qiagen #R0901) overnight at 4° C. and frozen thereafter at −20° C. For RNA preparation, samples (100 mg) are thawed and transferred to extraction tubes Lysing Matrix D 1,4 mm ceramic spheres (Fa. Mpbio #6913-500) for homogenization in 700 μL RLTplus buffer (Qiagen #1053393). Lysates are phenol-chloroform extracted and ⅓ is subjected to RNA isolation according to RNeasy® 96 Kit (Qiagen #74181) protocol. RNA yields are quantified and a constant amount of RNA is transcribed into cDNA with the use of High Capacity cDNA RT kit (Applied Biosystems, Cat #4368813). Gene expression levels are determined with the use of Quanti Fast Probe PCR Master Mix (Qiagen, Cat #204256) and respective Taqman Gene Expression Primer/Probes Assay on demand (Applied Biosystems). The markers are Col1a1 (Mm00801666_g1), Ctgf (Mm01192932_g1), Fap (Mm01329177_m1), Timp-1 (Mm00441818_m1), Itgam (Mm00434455_m1), Emr1 (Mm00802529_m1), Serpine1 (Mm00435860_m1), Saa1 (Mm00656927_g1). The marker ct-values of the single samples are compared to a standard curve of RNA mixture, generated from the respective experiment and resulting RNA quantity is normalized to 18S values (18S housekeeping gene, Applied Biosystems #4333760-1109036). Resulting normalized expression levels are divided by the mean of the control group and expressed as fold-change to control.

Statistical Tests

Statistical analyses are performed using Prism 4 Software (GraphPad Software, USA) using ONE-way ANOVA. P values<0.05 are considered statistically significant.

1. A pharmaceutical combination comprising:
(a) an AOC3 inhibitor of formula (I): 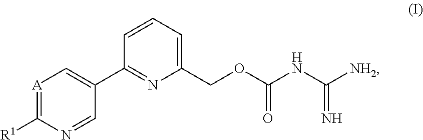
or a pharmaceutically acceptable salt thereof, wherein:
A is N or CH;
R¹ is selected from the group consisting of C₁₋₆-alkyl, C₃₋₆-cycloalkyl, heterocyclyl, —O—R², —S—R², —NH—R² and —N(R²)₂,
wherein each R² is independently selected from the group consisting of C₁₋₆-alkyl, C₃₋₆-cycloalkyl, heterocyclyl, —(C₁₋₂-alkyl)-(C₃₋₆-cycloalkyl), —(C₁₋₂-alkyl)-heterocyclyl, —(C₁₋₂-alkyl)-aryl, —(C₁₋₂-alkyl)-heteroaryl and —(C₁₋₂-alkyl)-C≡CH;
wherein each heterocyclyl of R¹ and R² is a 4- to 7-membered saturated carbocyclic group, in which 1 or 2 CH₂-moieties are independently of each other replaced by an atom or group selected from NH, O, S, —S(═O)—, —S(═O)₂— or —C(═O)—; and
wherein each aryl is selected from the group consisting of phenyl and naphthyl; and
wherein each heteroaryl is a 5- or 6-membered heteroaromatic ring which contains 1, 2 or 3 heteroatoms independently selected from ═N—, —NH—, —O— and —S—, wherein in heteroaromatic groups containing a —CH═N— unit, this group is optionally replaced by —NH—C(═O)—; and
wherein each alkyl, cycloalkyl, heterocyclyl, aryl or heteroaryl group of R¹ and R² is optionally independently substituted with one or more F, Cl, CN, OH, C₁₋₃-alkyl, —O—(C₁₋₃-alkyl), —C(═O)—(C₁₋₃-alkyl) and —C(═O)—(C₃₋₇-cycloalkyl);
wherein each of the above-mentioned alkyl groups may be linear or branched and are optionally substituted by one or more F; and
(b) an SGLT2 inhibitor.

2. The pharmaceutical combination according to claim 1 wherein the AOC3 inhibitor is of formula (I), or a pharmaceutically acceptable salt thereof, wherein:
A is N; and
R¹ is selected from the group consisting of cyclopropyl, heterocyclyl and —O—R²;
wherein R² is selected from the group consisting of C₁₋₆-alkyl, —(C₁₋₂-alkyl)-(C₃₋₆-cycloalkyl), —(C₁₋₂-alkyl)-heteroaryl and —(C₁₋₂-alkyl)-C≡CH;
wherein each heterocyclyl is selected from the group consisting of azetidinyl, piperidinyl, tetrahydrofuranyl, tetrahydropyranyl and morpholinyl; and
wherein each heterocyclyl group is optionally independently substituted with one substituent selected from F, CN, OH, CH₃, —O—CH₃; and
wherein each heteroaryl is selected from the group consisting of isoxazolyl, thiazolyl and thiadiazolyl; and
wherein each alkyl, cycloalkyl, heterocyclyl, or heteroaryl group is optionally independently substituted with one or more F, CN, CH₃, —OCH₃, —C(═O)—CH₃ and —C(═O)-cyclopropyl;
wherein each of the above-mentioned alkyl groups may be linear or branched and are optionally substituted by one or more F.

3. The pharmaceutical combination according to claim 1 wherein the AOC3 inhibitor is selected from the group consisting of 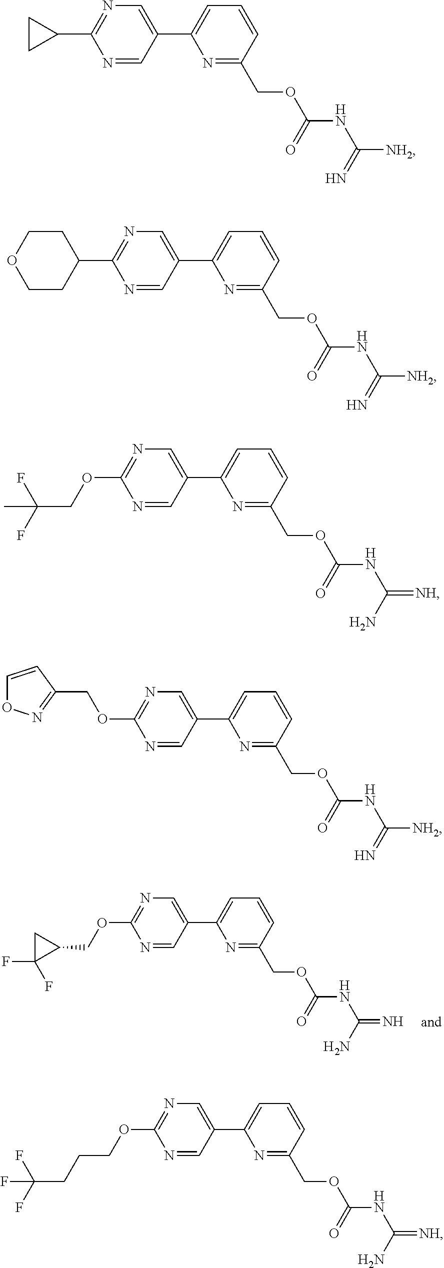 [SMILES: N=C(N)NC(=O)OCc1cccc(-c2cnc(C3CC3)nc2)n1 ; N=C(N)NC(=O)OCc1cccc(-c2cnc(C3CCOCC3)nc2)n1 ; CC(F)(F)COc1ncc(-c2cccc(COC(=O)NC(=N)N)n2)cn1 ; N=C(N)NC(=O)OCc1cccc(-c2cnc(OCc3ccon3)nc2)n1 ; N=C(N)NC(=O)OCc1cccc(-c2cnc(OC[C@H]3CC3(F)F)nc2)n1 ; N=C(N)NC(=O)OCc1cccc(-c2cnc(OCCCC(F)(F)F)nc2)n1]
or a salt thereof.

4. The pharmaceutical combination according to claim 1 wherein the SGLT2 inhibitor is selected from the group of empagliflozin, dapagliflozin, canagliflozin, ipragliflozin, tofogliflozin, luseogliflozin, atigliflozin, remogliflozin, sergliflozin, ertugliflozin and sotagliflozin.

5. A method for preventing, slowing the progression of, delaying or treating fibrotic disorders, metabolic disorders, inflammation disorders, ocular disease, neuroinflammatory disorders or cancer, the method comprising administering the pharmaceutical combination according to claim 1 to a patient in need thereof.

6. The method according claim 5 wherein the fibrotic disorder is selected from the group consisting of cystic fibrosis, interstitial lung disease, including idiopathic pulmonary fibrosis, liver fibrosis, non-alcoholic steatohepatitis (NASH), alcohol induced fatty liver, alcohol induced liver fibrosis, toxic fatty liver and cirrhosis of the liver, kidney fibrosis, scleroderma, radiation-induced fibrosis and other diseases where excessive fibrosis contributes to disease pathology.

7. The method according claim 5 wherein the metabolic disorder is selected from the group consisting of pre-diabetes mellitus, type 1 diabetes mellitus, type 2 diabetes mellitus, complications associated with diabetes mellitus, overweight, obesity, impaired glucose tolerance (IGT), impaired fasting blood glucose (IFG), hyperglycemia, postprandial hyperglycemia, insulin resistance, fatty liver, including non-alcoholic fatty liver disease (NAFLD), overweight, obesity and metabolic syndrome.

8. The method according claim 7 wherein the metabolic disorder is a complication associated with diabetes mellitus selected from the group consisting of cataracts and micro- and macrovascular diseases, such as diabetic nephropathy, glomerulosclerosis, diabetic retinopathy, choroidal neovascularisation, non-alcoholic fatty liver (NAFL) disease, non-alcoholic steatohepatitis (NASH), diabetic neuropathy, diabetic pain, tissue ischaemia, diabetic foot, diabetic ulcer, arteriosclerosis, myocardial infarction, accute coronary syndrome, unstable angina pectoris, stable angina pectoris, stroke, peripheral arterial occlusive disease, cardiomyopathy, heart failure, cardiovascular death, heart rhythm disorders and vascular restenosis.
